(19) United States
(12) Patent Application Publication
(10) Pub. No.: US 2025/0208135 A1
(43) Pub. Date: Jun. 26, 2025

(54) Biomarkers for Neurodegenerative Disease

(71) Applicant: Yeda Research and Development Co. Ltd., Rehovot (IL)

(72) Inventors: Michal Eisenbach-Schwartz, Rehovot (IL); Javier Maria Peralta Ramos, Rehovot (IL); Giulia Castellani, Rehovot (IL); Tommaso Croese, Rehovot (IL)

(73) Assignee: Yeda Research and Development Co. Ltd., Rehovot (IL)

(21) Appl. No.: 18/557,941

(22) PCT Filed: Apr. 28, 2022
(86) PCT No.: PCT/IL2022/050437

§ 371 Date: Oct. 29, 2023

Related U.S. Application Data
(60) Provisional application No. 63/180,801, filed on Apr. 28, 2021.

(51) Int. Cl. G01N 33/569; G01N 33/68

(52) U.S. Cl. CPC G01N 33/56972; G01N 33/6812; G01N 33/6896; G01N 2800/2821

(57) ABSTRACT

The present invention provides a method for early detection or diagnosis of a neurodegenerative disease, disorder, or condition in a subject at risk of developing or suspected of having the neurodegenerative disease, disorder, or condition, the method comprising measuring in a blood sample obtained from the subject or a fraction thereof the levels of at least one biomarker selected from CD38⁺ peripheral blood mononuclear cells (PBMCs), trigonelline, GLUT1 expression in CD4⁺ T cells, Tₕ2, Tₕ2/Tₕ1 ratio, naïve T cells, adenosine, allose, and HLA-DR T cells, as well as related methods and kits.

### FIELD OF THE INVENTION

The present invention relates in general to biomarkers and methods of using them for early detection and diagnosis of neurodegenerative diseases, such as Alzheimer's disease.

### BACKGROUND OF THE INVENTION

Alzheimer's disease (AD) is a progressive neurodegenerative disorder with a strong genetic component, and the most common cause of dementia in the elderly. Dominantly inherited AD, in which mutations in the amyloid precursor protein (APP), or in presenilin-1 or presenilin-2 results in a form known as early-onset AD, accounts for only 1% of the cases. Autosomal dominant mutations in individuals with familial AD are highly penetrant, with the onset of clinical symptoms occurring at a predictable age (typically between 30 and 50 years of age). Although several studies have recently proposed amyloid-beta (Aβ), tau, or neurofilament light chain (NfL) as candidate markers to assess changes in brain, current approaches are as yet unsuitable for routine clinical practice, with blood biomarkers for pre-symptomatic AD still lacking.

Healthy life-long brain plasticity depends on the integrity and function of the adaptive immunity, and the choroid plexus and meningeal T cell-derived cytokines, such as interferon-gamma, interleukin (IL)-17A, and IL-4. In animal models of familial Alzheimer's disease (FAD) low-grade chronic inflammation develops in the brain and the adaptive peripheral immune system develops a form of immune deficiency/immunosuppression with disease progression. In mouse models of AD, dysfunction of the immune system was seen prior to disease symptoms (Baruch et al. 2015b, Mesquita et al. 2015), and as a corollary, breeding 5×FAD mice with immune deficient mice was shown to exacerbate disease symptoms (Marsh et al. 2016).

Accordingly, the symptoms of neurodegenerative disease appear long after its onset and it is widely accepted that early intervention would be highly effective. However, there are currently no approved blood-based biomarkers and the presently known brain imaging techniques—especially magnetic resonance imaging (MRI) and positron emission tomography (PET) and the recently developed advanced PET tracers, such as Pittsburgh Compound-B (PiB) and Fluoro-2-deoxy-D-glucose (FDG)—are unable to provide reliable early detection of disease in asymptomatic persons.

There remains thus an urgent need for biomarkers for early detection of neurodegenerative disease, which potentially could support mechanisms underlying disease escalation.

### SUMMARY OF INVENTION

In one aspect, the present invention provides a method for early detection or diagnosis of a neurodegenerative disease, disorder, or condition in a subject at risk of developing or suspected of having the neurodegenerative disease, disorder, or condition, the method comprising measuring in a blood sample obtained from the subject or a fraction thereof the levels of at least one biomarker selected from CD38⁺ peripheral blood mononuclear cells (PBMCs), trigonelline, GLUT1 expression in CD4⁺ T cells, Tₕ2, Tₕ2/Tₕ1 ratio, naïve T cells, adenosine, allose, HLA-DR T cells,

wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that the subject is likely developing, or affected by, said neurodegenerative disease, disorder, or condition.

In a second aspect, the present invention provides a method for early detection or diagnosis of Alzheimer's disease (AD) in a subject at risk of developing or suspected of having AD, the method comprising measuring in a blood sample obtained from the subject or a fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells,

wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicate that the subject is likely developing, or affected by, AD.

In a third aspect, the present invention provides a method for stratifying a subject in a subgroup of a clinical trial of an immune checkpoint modulator for the treatment of a neurodegenerative disease, disorder, or condition, the method comprising measuring in a blood sample obtained from the subject or a fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells,
wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that that the subject is likely to be responsive to treatment with the immune checkpoint modulator, and
no change or a decreased level of at least one of CD38⁺ PBMCs, Tₕ2, Tₕ2/Tₕ1 ratio, naïve T cells, and adenosine, as compared to a respective reference, and/or no change or an increased level of at least one of trigonelline, GLUT1 expression in CD4⁺ T cells, allose, and HLA-DR T cells as compared to a respective reference, indicates that that the subject is likely not to be responsive to treatment with the immune checkpoint modulator, and said biomarker enables the stratification of said subject into a subgroup for said clinical trial.

In a fourth aspect, the present invention provides a method of assessing efficacy of a treatment by an immune checkpoint modulator in a subject diagnosed with a neurodegenerative disease, disorder, or condition, said method comprising:
obtaining a first blood sample from the subject;
administering the immune checkpoint modulator to the subject;
obtaining a second blood sample from the subject;
measuring in the first blood sample or a fraction thereof and in the second blood sample or fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells, and
comparing the level of the at least one biomarker in the first blood sample with the level of the biomarker in the second blood sample;
wherein a decreased level of at least one of CD38⁺ PBMCs, Tₕ2, Tₕ2/Tₕ1 ratio, naïve T cells, and adenosine, and/or an increased level of at least one of trigonelline, GLUT1 expression in CD4⁺ T cells, allose, and HLA-DR T cells in the second blood sample compared to the first blood sample indicates that the treatment by an immune checkpoint modulator is likely effective.

In a fifth aspect, the present invention provides a method for treating or preventing a neurodegenerative disease, disorder, or condition in a subject, said method comprising measuring in a blood sample or fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells,
wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that the subjects is likely developing, or is affected by, the neurodegenerative disease, disorder, or condition, and
the method further comprising administering an immune checkpoint modulator to the subject which is likely developing, or is affected by, the neurodegenerative disease, disorder, or condition, thereby treating the subject.

In a sixth aspect, the present invention provides a kit comprising at least one antibody specific for CD3, at least one antibody specific for CD4, and at least one antibody specific for CD38; and at least one antibody specific for a biomarker selected from the following biomarkers: CXCR5, CCR6, CXCR3, CCR7, CD45RO, CD45RA, CD8, CCR7, CD45RO, CD45RA, CD19, CD27, CD11c, CD14, CD16, IFNγ, GLUT1, HLA-DR, IL-10, IL-22, and IL-4.

### BRIEF DESCRIPTION OF THE DRAWINGS

FIGS. 1A-1E. APP familial AD individuals present a T_{H}2 disease-associated immune profile before symptoms onset. PBMCs from healthy (n=18, full black circles) and pre-symptomatic AD (n=16, empty diamonds) individuals were analyzed by mass cytometry. A. Frequency of CD4+ and CD8+ T cells, out of total CD45+ immune cells. B. Frequency of T_{H}1, T_{H}2 and T_{H}17 cells, out of CD4⁺ Tₕ cells. C. Frequency of activated HLA-DR⁺ T cells, out of CD4⁺ T cells. D.

Frequency of T_{Naïve}, T central memory_{(CM)}, T effector memory_{(EM)}, and T terminally differentiated effector memory_{(EMRA)} cells, out of CD4⁺ T cells. E. Frequency of T_{Naïve}, T_{CM}, T_{EM}, and T_{EMRA} cells, out of CD8⁺ T cells. F. Concentration of plasma neurofilament light chain (NfL) across all sample groups assessed. P values are based on multiple Mann-Whitney U-tests, without correction for multiple comparisons. *P<0.05, **P<0.01, ***P<0.001. All data show the mean±s.e.m.

FIGS. 2A-2F. Differential expression of the age-associated CD38 ecto-enzyme precedes symptoms onset in familial AD T cells. PBMCs from healthy (n=18, full black circles) and pre-symptomatic AD (n=16, empty diamonds) individuals were analyzed by mass cytometry. A, B. Frequency of CD38-expressing cells (left: CD38⁺; middle; CD38^{hi}; right: CD38^{lo}), out of total CD4⁺ (A) and CD8⁺ (B) T cells. C, D. Standard deviation of CD38 in CD4⁺ (C) and CD8⁺ (D) T cells. E, F. Single, live CD4⁺ (E) and CD8⁺ (F) T cells from healthy (n=11) and pre-symptomatic AD (n=9-13) individuals were selected and further analyzed by mass cytometry. Normalized and scaled IL-10 and IL-22 median expression (MdE) by CD4⁺ T_{H} (E) and IL-4 by CD8⁺ (F) T cells, respectively, is shown. P values are based on two-tailed Mann-Whitney U-test. *P<0.05, **P<0.01, ***P<0.001. All data show the mean±s.e.m.

FIGS. 3A-3D. Pre-symptomatic AD individuals feature a selective metabolic impairment in CD4⁺ T cells. Single, live CD4⁺ and CD8⁺ T cells from healthy (n=11) and pre-symptomatic AD (n=9-13) individuals were selected and further analyzed by mass cytometry. A, B. Principal component analysis (PCA), based on all immune-metabolic markers used (see Table 1), is shown for both single, live CD4⁺ (A) and CD8⁺ (B) T cells. Inset shows pairwise permutational analysis of variance (PERMANOVA). C, D. Normalized and scaled surface median expression (MdE) of the glucose receptor GLUT1 on CD4⁺ Tₕ (C) and CD8⁺ (D) T cells, respectively, is shown. P values are based on two-tailed Mann-Whitney U-test. **P<0.01. All data show the mean f s.e.m.

FIGS. 4A-4G. APP familial AD individuals exhibit a distinct plasma metabolome. A. Volcano plot of significantly enriched plasma metabolites in healthy (n=14, filled black circles) vs. pre-symptomatic AD (n=13, empty diamonds) individuals, as determined by untargeted mass spectrometry metabolomic profiling. Significance according to an FDR-corrected two-tailed Mann-Whitney U-test. B, C. Significantly modulated plasma metabolites (arbitrary units, AU): allose (B) and trigonelline (C), in healthy as compared to pre-symptomatic (AD) individuals. D-G. Selected plasma metabolites for targeted mass spectrometry metabolomic quantification and validation: nicotinamide (D), trigonelline (E), Adenosine diphosphate ribose (ADPR, F), adenosine (G). B-G. P values are based on two-tailed Mann-Whitney U-test. *P<0.05, **P<0.01. All data show the mean±s.e.m.

### DETAILED DESCRIPTION

In order to determine a molecular immune cell signature across Alzheimer's disease (AD) progression, the inventors used in-depth immune profiling of human-derived blood samples, from a cohort of individuals who carry an autosomal dominant amyloid precursor protein (APP) duplication, and were either pre-symptomatic (indicated as “AD” in the figures, n=16), clinically diagnosed with early-onset familial AD (n=4), or individuals in which genetic testing did not show the APP gene duplication (healthy, n=18). Because of the small number of the symptomatic individuals, they were not included in the analysis.

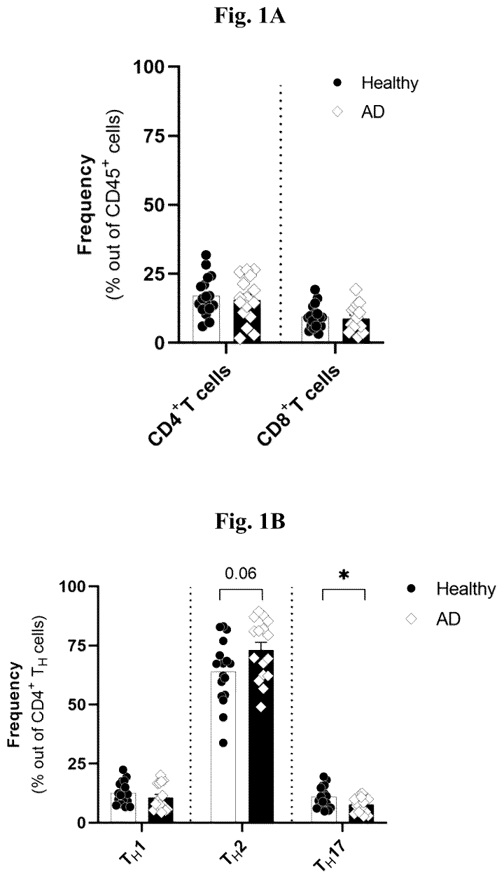

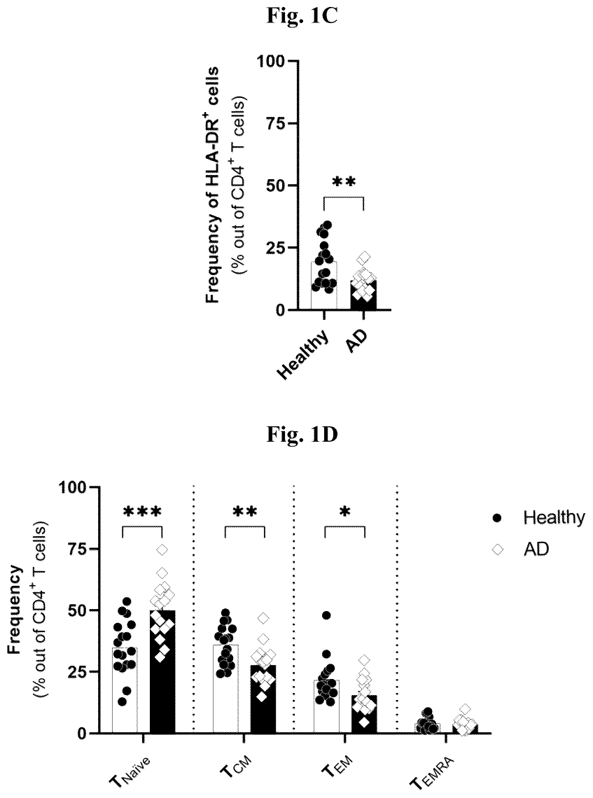

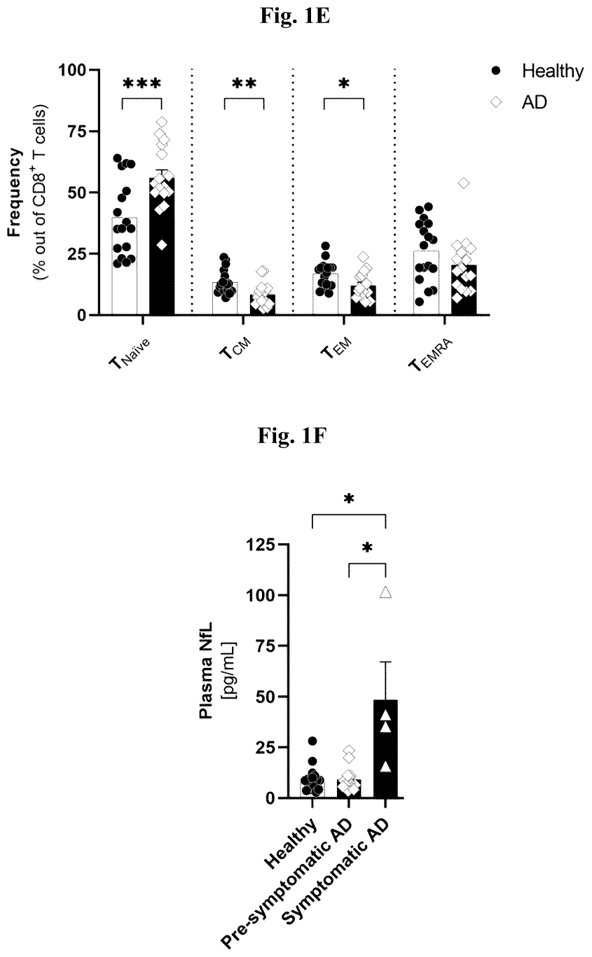

Proteomic phenotyping of the blood immune landscape was conducted by high-dimensional single-cell mass cytometry (CyTOF) on peripheral blood mononuclear cells (PBMCs), encompassing identity, function, and immune regulation of peripheral blood mononuclear cells to determine their cellular, metabolic and cytokine polarization immune profile (FIGS. 1A-1E) (Hartmann et al., 2019).

The inventors have found a previously undescribed T helper 2 (T_{H}2)-disease-associated immune fingerprint in pre-symptomatic AD individuals (i.e., preceding the onset of AD symptoms), as shown by CyTOF (mass cytometry) (FIG. 1B), and reduction of the frequency of HLA-DR⁺ CD4⁺ T cells in the pre-symptomatic group (FIG. 1C).

Disease state was assessed by neuronal loss measured as levels of neurofilament light chain (NfL) level in plasma of pre-symptomatic patients relative to the 4 patients in our study and a cohort of unrelated patients that were diagnosed as affected, based on mini-mental state examination (MMSE). The results confirmed that the pre-symptomatic individuals were indeed prior to clinical manifestation of disease with no elevation of NfL in the blood, in contrast to those with diagnosed AD (FIG. 1F). Further analysis of the T cells at different differentiation stage, including naïve, effector memory (T_{EM}), central memory (T_{CM}), and terminally differentiated effector memory (T_{EMRA}), revealed that in both CD4⁺ and CD8⁺, the subpopulations that were most affected included the naïve T cells, which were elevated, and the T_{CM}, which were reduced (FIGS. 1D, 1E). These effects were more pronounced in the CD4⁺ memory T cells than in the CD8⁺ memory cells.

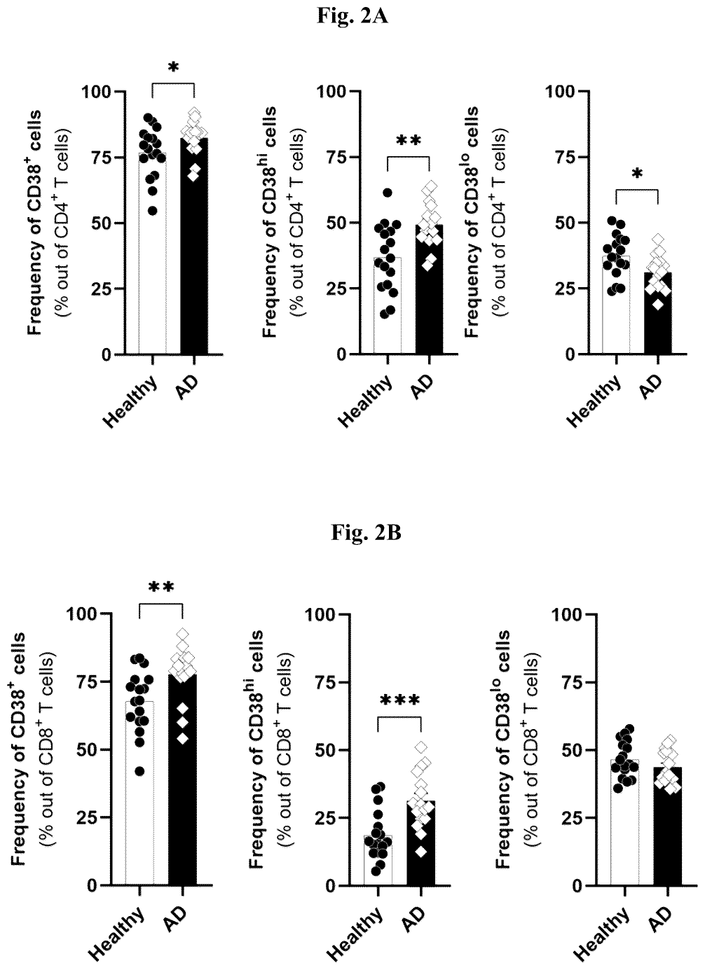

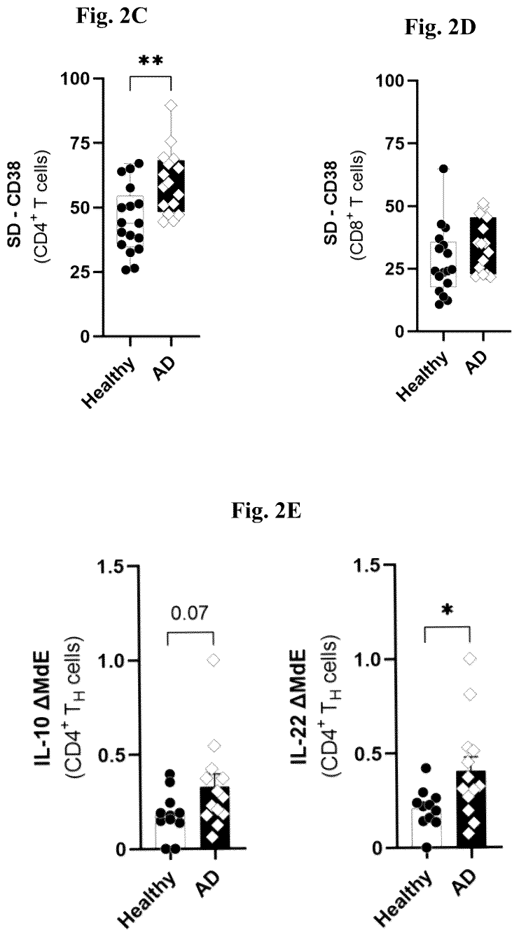

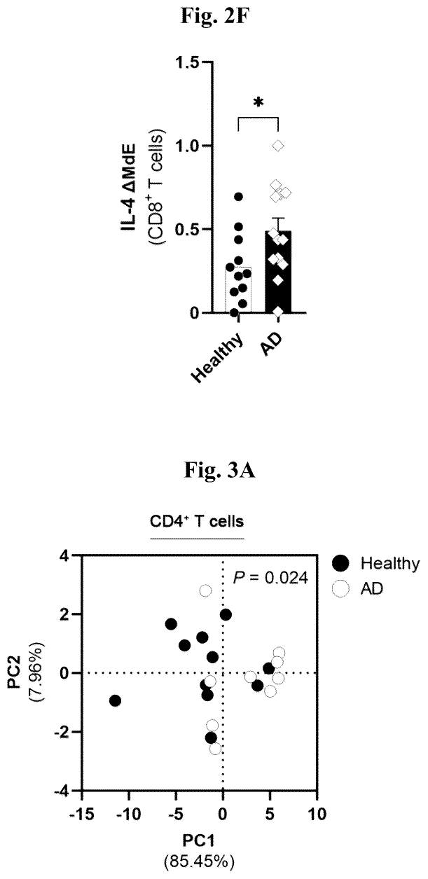

The inventors also screened for expression of CD38, which is a multifunctional ecto-enzyme (a catalytic membrane protein with the active site outside the cell) that metabolizes NAD+ and mediates nicotinamide dinucleotide (NAD⁺) and extracellular nucleotide homeostasis as well as intracellular calcium. The results show an elevation in CD38^{hi} out of total CD4⁺ T cells or total CD8⁺ T cells in the pre-symptomatic patients (FIGS. 2A and 2B, respectively). Overall, a positive correlation was found between levels of T_{H}2 cells as defined by cellular markers CXCR5⁻CCR6⁻CXCR3⁻CD4⁺ and levels of CD38 cells across all APP mutation carriers (data not shown).

To assess the cytokine and transcription factor (TFs) polarization profile of T cells from affected and pre-symptomatic family members, the inventors carried out a comprehensive single-cell immunophenotyping protocol. An increase in IL-10 and IL-22-expressing CD4⁺ cells, and IL-4-expressing CD8⁺ T cells was observed in pre-symptomatic individuals, validating the previously observed type 2 Tₕ response (FIGS. 2E, 2F). In light of these results and given the low number of symptomatic patients, subsequent analyses focused on the pre-symptomatic patients, striving to understand the mechanism responsible for these changes.

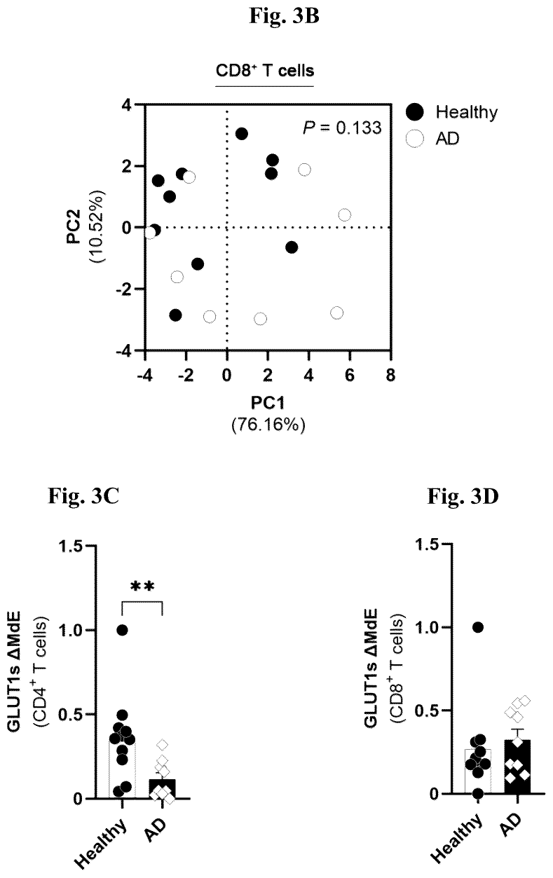

Because of the association of CD38 expression with metabolic activity, the next step was to focus on possible metabolic changes in the patients with AD-associated mutations. CD38 catalyzes the synthesis of nicotinamide (NAM) and adenosine diphosphate ribose (ADPR) using nicotinamide adenine dinucleotide (NAD⁺) as a substrate. NAD⁺ is an essential cofactor that regulates energy metabolism. Metabolic rewiring of T cells has been shown to dictate cell function. Therefore, to understand metabolic changes in T cells of patients destined to develop AD, a multiplexed metabolic profiling pipeline for T cells was developed, targeting key NAD⁺-consuming enzymes, glycolytic proteins, TCA/ETC components, markers of mitochondrial dynamics, and signaling/transcription molecules/factors, in order to assess their overall immuno-metabolic state. Interestingly, CD4⁺ but not CD8⁺ T cells derived from pre-symptomatic individuals revealed a striking and selective metabolic impairment, particularly a decrease in the surface expression level of the glucose transporter 1 (GLUT1) (FIGS. 3C, 3D), suggesting distinct cell-intrinsic metabolic programs for CD4⁺ and CD8⁺ T cells before symptom onset in dominantly inherited AD. These results suggest metabolically dysfunctional CD38-expressing CD4⁺ T cells as potential players driving a T_{H}2-disease-associated immune signature that precedes symptom onset in carriers of dominantly inherited AD.

Recent findings attributed a key role to metabolic programs in shaping effective immune responses, instructing T cell fate (Buck et al. 2016) and driving T helper (T_{H}) lineage commitment (Arbore et al. 2016, Puleston et al. 2021). Moreover, senescence-induced inflammation has been strongly associated with NAD-consuming enzymes, such as CD38 (Camacho-Pereira et al., 2016, Tarragó et al. 2018, Chini et al. 2020), thereby driving age-related NAD⁺ decline and mitochondrial dysfunction. Yet, the nature and functional role of circulating T cells in AD remains to be fully elucidated.

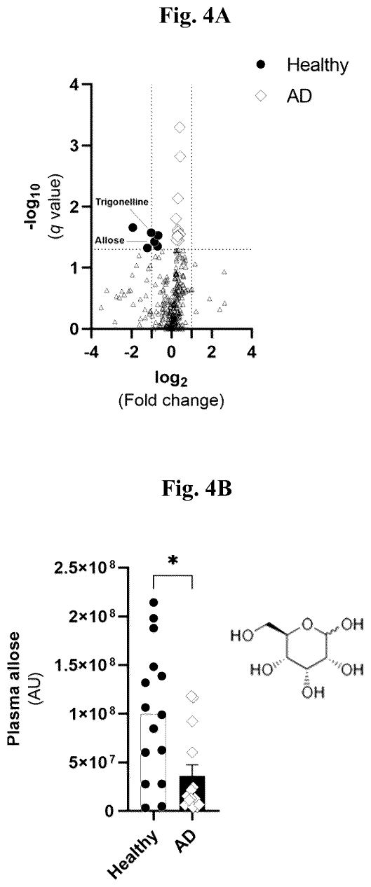

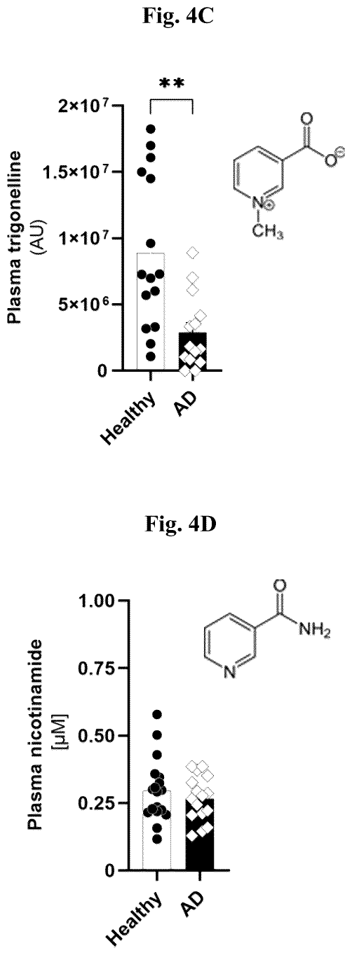

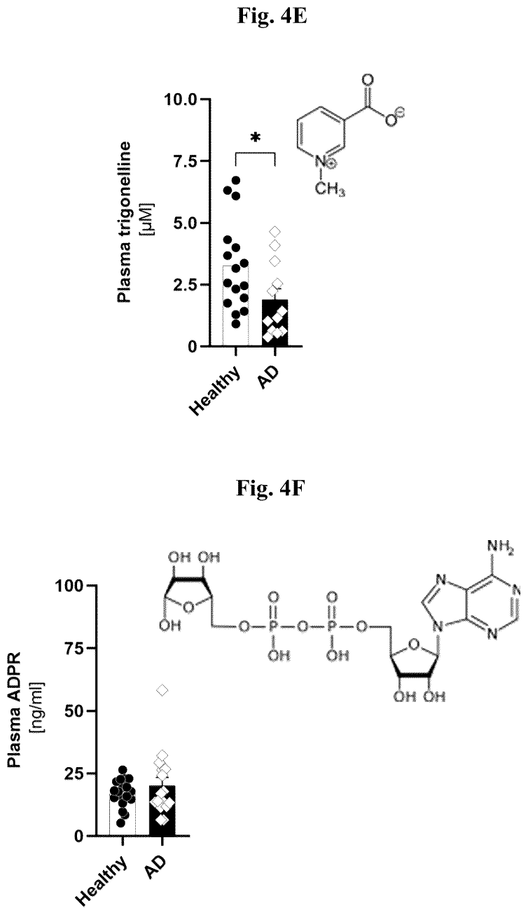

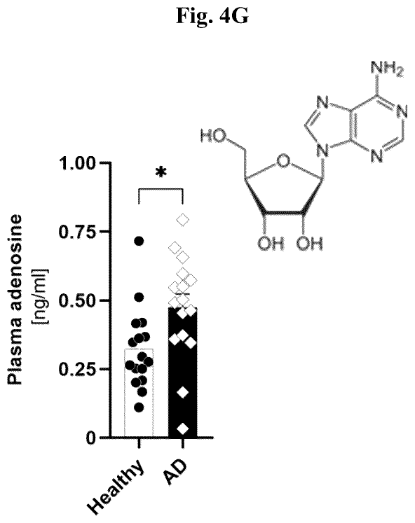

As noted above, characterization of the T cell compartment revealed an increase in naïve CD4⁺ T cells, together with a decrease in effector CD8⁺ T cells (T_{EM}, FIG. 1E) in AD subjects, as compared to their age-matched healthy counterparts. Further in silico proteomic analysis together with untargeted metabolic profiling (FIGS. 4A-4G) led to the identification of a previously undescribed expansion of peripheral CD38-expressing T cells and a remarkable reduction in trigonelline (N-methylnicotinate) in pre-symptomatic individuals (FIGS. 4C, 4E), suggesting a potential metabolic dysfunction in the NAD⁺ biosynthetic pathway before symptom onset in familial AD. Targeted metabolomics also revealed an elevation in adenosine (FIG. 4G), which is known to be an inhibitory metabolite, and a reduction in allose (a major metabolite of fructose/mannose, FIG. 4B), and in 3-(3-hydroxyphenyl)propionic acid (a major metabolite of phenylalanine, data not shown).

To summarize, the inventors discovered changes in adaptive immune cells in pre-symptomatic individuals who carry a mutation in APP. The overall changes were manifested in both CD4⁺ and CD8⁺ T cells, though they were more robust in the CD4⁺ cells. The changes were manifested by elevation of circulating naïve T cells and reduction in memory T cells that were more pronounced in the CD4⁺ cells. In addition, within the CD4⁺ T cells, skewing towards T_{H}2 type cells was found in the pre-symptomatic patients, correlating with expression of CD38.

Inverse relationships were found between levels of GLUT1 and CD38 within the CD4⁺ T cell population.

The changes observed in the young APP carriers prior to symptom onset can be explained in light of what we know about the fate of circulating T cells in general, and in aging in particular. Immune ageing is associated with numerous changes in immune cell subsets, antigen-specific cells and cytokines, consistent with an increasing acquisition of a T_{H}2-cell bias. Here, the inventors found that changes in the immune system that preceded symptomatic disease in patients that carry a mutation leading to a familial form of AD, are also manifested by skewing towards T_{H}2, associated with expression of CD38 and reduction in GLUT1 receptor expression. The early dysfunction of the immune system might be driven the chronic low-grade-inflammation in the brain in the carriers, a common phenomenon in immuno-aging.

The increased levels of naïve and reduction in memory T cells observed here could be driven by a metabolic deficit. Naïve T cells are largely quiescent and have relatively low energy demands. Recent studies suggest that GLUT1 expression is not required for peripheral survival of naïve T cells. Memory T cells, in contrast, must respond rapidly upon secondary encounter with their cognate antigen. To facilitate this rapid response, memory T cells engage the early shift towards aerobic glycolysis more rapidly than naïve cells, which is thought to support rapid production of cytokines such as IFN-γ. Upon activation, T cells proliferate and differentiate into effector T cells. This transition requires an increase in energy generation. This could explain the lack of shift towards differentiation. It is possible that the shift is also driven by an increase in adenosine in the circulation, which is known to inhibit T cells.

Altogether, the inventors present metabolically dysfunctional CD38-expressing T cells as potential players driving the Tₕ2-disease-associated immune signature that precedes symptoms onset in AD individuals. In fact, these changes represent the point in time, during a neurodegenerative process, in which the dormant disease or progressive damage traverses the point of no return to an active progressive neurodegenerative disease.

Accordingly, this T cell subset, characterized by increased expression of CD38, together with the decreased plasma metabolites, may be used as blood-based prognostic and/or diagnostic biomarkers not only for AD in familial patients, but also in sporadic patients at risk, and even in subjects in the process of developing neurological disease, either as a result of central nervous system (CNS) injury, or subjects which show early signs of dementia, or another motor or cognitive dysfunction.

Importantly, the inventor's approach and findings presented herein in AD patients, do not directly target any disease-specific factor in AD, such as amyloid beta or tau pathology, but rather demonstrate a principle that now stands on solid scientific ground, and which is expected to be clinically applicable in a wide range of CNS pathologies. This principle is centered around the interplay between the blood-cerebrospinal fluid barrier (BCSFB) and various immune cells.

The BCSFB is formed by the choroid plexus and functions as a specialized neuroimmunological interface between the brain and the blood circulatory system. In contrast to the blood-brain barrier (BBB), which under healthy conditions does not enable leukocyte trafficking, the choroid plexus also serves as a selective gateway for leukocytes entering the central nervous system (CNS), both for normal physiological immune surveillance of the CNS, and following brain or spinal cord tissue damage. Located in all four brain ventricles, the choroid plexus comprises fenestrated blood capillaries surrounded by a monolayer of epithelial cells interconnected by tight junctions. The choroid plexus stroma, the space between endothelium of the blood vessels and the epithelial monolayer, is populated by various immune cell types, including CD4⁺ T effector memory (T_{EM}) with antigen specificity for CNS antigens, associated with “protective autoimmunity” (Baruch and Schwartz, 2013; Baruch et al., 2013; Schwartz and Baruch, 2014).

Under physiological conditions, the ongoing presentation of CNS-derived antigens and continuous stimulation of the local T_{EM} cells within the choroid plexus stroma establishes a neuroimmunological interface that serves as a site of constant brain-immune dialogue. This dialogue supports CNS function by mediating CNS immune surveillance, controlling leukocyte trafficking into the CNS, maintaining neurotropic factor production by the choroid plexus and integrating signals from the brain with signals coming from the circulation. The BCSFB is an immunologically active surface well-equipped to mediate controlled immune cell trafficking and strategically positioned to sense and respond to signals from compartments that separate the blood and the CNS.

One way by which the BCSFB mediates this dialogue is through the interferon-gamma (IFN-γ) signaling cascade (Baruch et al., 2014, 2015a; Kunis et al., 2015). Under physiological conditions, an immune response initially increases the availability of IFN-γ at the secondary lymphoid organs (lymph nodes, spleen, etc.). This immune response leads to IFN-γ production by immune cells located within the stroma of the choroid plexus. Release of IFN-γ stimulates the choroid plexus epithelium to express leukocyte trafficking molecules including adhesion molecules and chemokines, resulting in selective transepithelial migration of leukocyte across the choroid plexus epithelium into the cerebrospinal fluid. This migration facilities the recruitment of T cells, and circulating monocytes which once within the CNS territory are regarded as monocyte-derived macrophages, distinct from the resident microglia. In fact, even under steady-state conditions, basal IFN-γ-dependent choroid plexus activity enables entry of a small number of leukocytes to the CNS (Baruch et al., 2015a; Kunis et al., 2013).

Under pathologic conditions, such as, e.g., brain aging (Baruch et al., 2013, 2014) and chronic neurodegenerative diseases (Baruch et al., 2015b, 2016; Kunis et al., 2015), multiple mechanisms are triggered that counteract IFN-γ signaling, and these contribute to disease pathology by excessively inhibiting supportive entry of immune cells to the CNS. For example, a decrease in IFN-γ-producing Tₕ1 cells was observed at the choroid plexus of ALS (Kunis et al., 2015) and AD mouse models (Baruch et al., 2015b, 2016), while brain aging is accompanied by increased systemic and choroid plexus levels of Treg cells, as well as of systemic myeloid-derived suppressor cells (MDSCs), and enhanced local Tₕ2 signaling at the choroid plexus (Baruch et al., 2013, 2014).

Whatever the cause for the low availability of IFN-γ at the choroid plexus under these pathological conditions, the end result is that it impairs brain-immune dialogue that is necessary for supporting brain tissue repair. Thus, systemic immune suppression, which is reflected in the changes in the level of the markers identified according to the present invention, may curtail the ability to mount the protective, cell-mediated immune responses that are needed for brain maintenance and repair.

Accordingly, in one aspect, the present invention provides a method for early detection or diagnosis of a neurodegenerative disease, disorder, or condition in a subject at risk of developing or suspected of having the neurodegenerative disease, disorder, or condition, the method comprising measuring in a blood sample obtained from the subject or a fraction thereof at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR⁺ T cells, wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that the subject is likely developing, or affected by, said neurodegenerative disease, disorder, or condition.

In some embodiments, an equal level or a decreased level of at least one of CD38⁺ PBMCs, Tₕ2, Tₕ2/Tₕ1 ratio, naïve T cells, and adenosine compared with the respective reference, or an equal or increased level of at least one of trigonelline, GLUT1 expression in CD4⁺ T cells, allose, and HLA-DR T cells compared with the respective reference, indicates that the subject is not likely developing, or is not likely affected by, said neurodegenerative disease, disorder, or condition.

The term “early detection” as used herein means detection in pre-symptomatic individuals, or individuals not yet having full-blown symptoms which enable the clear diagnosis of a known neurodegenerative disease, disorder, or condition.

The term “diagnosis” as used herein means to determine or to aid in determining whether the subject is affected by a known neurodegenerative disease, disorder, or condition. The methods of the present invention are not necessarily intended to be used as a sole method for diagnosing a specific neurodegenerative disease, disorder, or condition, and may be used together with additional methods.

The term “subject at risk of developing a neurodegenerative disease, disorder, or condition” as used herein refers to an asymptomatic (for the respective disease) subject (individual) who belongs to a risk group for developing the neurodegenerative disease, disorder, or condition for any reason. For example, one reason may be genetic, or family-related, namely that the subject is either a known carrier of a genetic aberration known to be associated with the neurodegenerative disease, disorder, or condition, or has family members manifesting the neurodegenerative disease, disorder, or condition and therefore the subject may be a carrier.

Other reasons for belonging to a risk group include, for example, advanced age, associated diseases, and symptoms indicative of a cognitive decline. A subject at risk of developing a neurodegenerative disease, disorder, or condition, may also be a subject having suffered brain or central nervous system (CNS) injury.

For example, a subject at risk for AD may be a person of above 60 years of age, or having a genetic aberration related to familial AD. For example, genes known to be associated with Alzheimer's disease are the genes encoding amyloid protein precursor (APP), presenilin-1 (PSEN1), a subunit of γ-secretase, the aspartyl protease responsible for AD generation, and presenilin-2 (PSEN2), the highly homologous gene of PSEN1. Mutations in the presenilin genes are the most common cause of familial Alzheimer's disease. Mutations in, and duplications of, the APP gene are also known as familial Alzheimer's disease risk factors. Thus, a non-limiting example of a subject at risk of developing a neurodegenerative disease, disorder, or condition, specifically AD, is a subject having a genetic aberration associated with the APP or PSEN genes.

In some embodiments, the subject is a human subject. In some embodiments, the subject is at risk for developing AD.

The term “genetic aberration” as used herein when referring to APP, refers to either an APP mutation or an APP gene duplication; when referring to PSEN, the term refers to a PSEN mutation.

The term “subject suspected of having the neurodegenerative disease, disorder, or condition” as used herein refers to a subject exhibiting early or mild symptoms of motor or cognitive dysfunction. The symptoms exhibited by the subject may not be sufficient for diagnosing a particular neurodegenerative disease, but may imply the need for intervention.

Early or mild symptoms of cognitive dysfunction as referred to here are also known as Mild Cognitive Impairment (MCI), which refers to patients who have memory or cognitive impairment but without obvious influence on their daily activities. The Mini-Mental State Examination (MMSE), Montreal Cognitive Assessment (MoCA), and Mini cognitive scale (Mini-Cog) are the most commonly used cognitive function screening scales used today.

For example, guidelines for diagnosing three stages of Alzheimees disease have been published by the US Alzheimer's Association and the National Institute on Aging (NIA): (1) dementia due to Alzheimees, (2) mild cognitive impairment (MCI) due to Alzheimees, and (3) preclinical (presymptomatic) Alzheimer's (Clifford R. Jack Jr. et al. “Introduction to the recommendations from the National Institute on Aging—Alzheimees Association workgroups on diagnostic guidelines for Alzheimees disease.” Alzheimer's & Dementia: The Journal of the Alzheimer's Association 2011; 7(3):257-262; Guy M. McKhann et al. “The diagnosis of dementia due to Alzheimees disease: Recommendations from the National Institute on Aging —Alzheimer's Association workgroups on diagnostic guidelines for Alzheimees disease.” Alzheimer's & Dementia: The Journal of the Alzheimer's Association 2011; 7(3):263-269; Marilyn S. Albert et al. “The diagnosis of mild cognitive impairment due to Alzheimer's disease: Recommendations from the National Institute on Aging—Alzheimer's Association workgroups on diagnostic guidelines for Alzheimer's disease.” Alzheimer's & Dementia: The Journal of the Alzheimer's Association 2011:7(3):270-279; Reisa A. Sperling et al. “Toward defining the preclinical stages of Alzheimer's disease: Recommendations from the National Institute on Aging—Alzheimer's Association workgroups on diagnostic guidelines for Alzheimer's disease.” Alzheimer's & Dementia: The Journal of the Alzheimer's Association 2011; 7(3):280-292.)

Early or mild symptoms of motor disease may include muscle weakness, loss of balance, muscle atrophy, cramps and fasciculations, pathological reflexes. Additionally, any symptom that is commonly considered to be an early possible sign of a motor-neuron diseases is meant to be included.

The term “APP mutation carrier” as used herein refers to a person carrying an APP mutation known to be associated with Alzheimer's disease and Cerebral Amyloid Angiopathy or to cause increased formation of amyloidogenic AD peptides, e.g. selected from the list in https://www.alzforum.org/mutations/app; or to a person having an APP gene duplication.

The term “PSEN mutation carrier” as used herein refers to a person carrying a presenilin-1 (PSEN1) or a presenilin-2 (PSEN2) mutation, such as one of the more than 300 known mutations in PSEN1 and one of the more than a dozen known mutations in PSEN2.

Methods for detecting familial Alzheimer's disease in asymptomatic subjects are also well-known in the art. For example, PCR-based methods can be performed for monitoring the mutations in the AD risk factor genes. Genomic DNA can be extracted from total blood, buffy coat (white blood cells), bone marrow, or cell cultures, using a specific extraction kit. DNA should be amplified by specific primers, designed for the AD risk-factor genes (see below). Several mutation detection methods have been developed, such as restriction fragment length polymorphism (RFLP), single-strand conformation polymorphism (SSCP), denaturing gradient gel electrophoresis (DGGE), temperature gradient gel electrophoresis (TGGE), and heteroduplex analysis (Bagyinszky, Eva & Youn, Young Chul & An, Seong & Kim, SangYun. (2014). The genetics of Alzheimer's disease. Clinical interventions in aging. 9. 535-551).

Furthermore, recently Giau et al. (Giau, V. V., Bagyinszky, E., Yang, Y. et al. Genetic analyses of early-onset Alzheimer's disease using next generation sequencing. Sci Rep 9, 8368 (2019). https://doi.org/10.1038/s41598-019-44848-2) reported that in addition to the known mutations, three new missense mutations in PSEN1 (T119I, G209A, and G417A) were discovered by next generation sequencing and 67 missense mutations in susceptibility genes for late-onset AD were identified, which may be involved in cholesterol transport, inflammatory response, and β-amyloid modulation. This group also identified 70 additional novel and missense variants in other genes, such as MAPT, GRN, CSFIR, and PRNP, related to neurodegenerative diseases, which may represent overlapping clinical and neuropathological features with AD. Extensive genetic screening of Korean patients with early-onset Alzheimer disease identified multiple rare variants with potential roles in AD pathogenesis. Other genes that are or may be risk factors for AD are apolipoprotein E (APOE) E4 allele, clusterin (CLU), complement receptor 1 (CR1), phosphatidylinositol binding clathrin assembly protein (PICALM), and sortilin-related receptor (SORL 1). Recent studies have discovered additional novel genes that might be involved in late-onset AD, such as triggering receptor expressed on myeloid cells 2 (TREM2) and cluster of differentiation 33 (CD33) (Bagyinszky E, Youn Y C, An S S, Kim S. The genetics of Alzheimer's disease. Clin Interv Aging. 2014; 9:535-551).

In certain embodiments, the subject at risk of developing or suspected of having, the neurodegenerative disease, disorder, or condition is a person over 60 years of age.

The term “biomarker” as used herein, relates to a parameter that changes between healthy controls, and disease carriers (pre-symptomatic) or active patents (symptomatic), and this change may be used to diagnose or predict disease. Some examples for a biomarker may be a level of a certain cellular marker or cell type or a certain molecule in plasma or serum, a ratio between a certain cell type and another cell type, and a frequency of a specific cell type out of a more general cellular population. The change may be an increased level of the specific biomarker compared to a respective reference such as the levels in healthy population, or a decreased level of the specific biomarker compared to a respective reference such as the levels in healthy population

In general, a biomarker is considered increased or decreased if the levels of the biomarker in the suspected individual are increased or are decreased compared to the respective reference by at least about 10%, 15%, 20%, 25%, 30%, 35%, 40%, 45%, 50%, 60%, 70%, 80%, 90%, or 100%.

The cell types referred to herein are defined by cellular biomarkers as follows:

T cells are identified by the markers: CD45⁺ CD3⁺.

CD4⁺ T cells are identified by the markers: CD45⁺CD3⁺CD4⁺. In some embodiments, CD4⁺ T cells are identified by measuring CD4 alone. In some embodiments, CD4⁺ T cells are identified by measuring CD4 as well as the more general CD3 and/or CD45.

CD8⁺ T cells are identified by the markers: CD45⁺ CD3⁺CD8⁺. In some embodiments, CD8⁺ T cells are identified by measuring CD8 alone. In some embodiments, CD8⁺ T cells are identified by measuring CD8 as well as the more general CD3 and/or CD45.

T helper (Tₕ) cells are CD4⁺ CXCR5⁻ T cells.

Tₕ1 cells are identified by the markers: CD45⁺CD3⁺CD4⁺CXCR5⁻CCR6⁻CXCR3⁺, and are also identified herein as CXCR5⁻CCR6⁻CXCR3⁺ CD4⁺ cells, or CCR6⁻CXCR3⁺ Tₕ cells. Tₕ1 cells are functionally (at least) IFN-γ-producing cells.

In some embodiments, the method further comprises measuring in a blood sample obtained from the subject or a fraction thereof the level of Tₕ1 cells.

Tₕ2 cells are identified by the markers: CD45⁺ CD3⁺CD4⁺CXCR5⁻CCR6⁻CXCR3⁻, and are also identified herein as CXCR5⁻CCR6⁻CXCR3⁻CD4⁺ cells, or CCR6⁻CXCR3⁻ Tₕ cells. Tₕ2 cells are functionally (at least) IL-4 and IL-10-producing cells.

Tₕ17 cells identified by the markers: CD45⁺ CD3⁺CD4⁺CXCR5-CCR6⁺CXCR3⁻, and are also identified herein as CXCR5-CCR6⁺CXCR3⁻ CD4⁺ cells, or CCR6⁺CXCR3⁻ Tₕ cells. Tₕ17 cells are functionally (at least) IL-17 and IL-22-producing cells.

In some embodiments, the method further comprises measuring in a blood sample obtained from the subject or a fraction thereof the level of Th17 cells.

CD38⁺ PBMCs may be any type of blood mononuclear cells, including, e.g., T lymphocytes, B lymphocytes, NK cells, monocytes, or any other blood mononuclear cell expressing the CD38⁺ marker.

CD38⁺ T cells are identified herein as CD4⁺CD38⁺ T cells or CD8⁺CD38⁺ T cells, respectively. In some embodiments, CD38⁺ T cells are identified by the markers: CD45⁺ CD3⁺CD4⁺CD38⁺ or CD45⁺CD3⁺CD8⁺CD38⁺. In some embodiments, CD38⁺ T cells are identified by the markers: CD3⁺CD4⁺CD38⁺ or CD3⁺CD8⁺CD38⁺. In some embodiments, CD38⁺ T cells are identified by the markers: CD4⁺CD38⁺ or CD8⁺CD38⁺.

In some embodiments, CD38 cells are CD4⁺ T cells. In some embodiments, CD38 cells are CD8⁺ T cells. In some embodiments, CD38 cells are classical monocytes. In some embodiments, CD38 cells are plasma cells or B cells.

CD38^{hi} cells are CD38 cells as defined above, which have an increased level of the CD38 molecule per cell.

CD38^{lo} cells are CD38 cells as defined above, which have a decreased level of the CD38 molecule per cell.

In other words, CD38^{hi} cells and CD38^{lo} cells are subsets of CD38⁺ cells, having a higher or a lower density of the CD38 antigen, respectively.

In some embodiments, the CD38^{hi} cells and CD38^{lo} cell populations are distinguished by gating of the populations—both expressing CD4 or CD8 and CD38, but one population with high CD38 expression and the other population with low CD38 expression, as can be seen and separated by a mass cytometry method.

Naïve CD4/CD8⁺ T cells (T_{Naïve}) are identified by the markers: CD45⁺CD3⁺CD4⁺CCR7CD45RO⁻CD45RA⁺ or CD45⁺CD3⁺CD8⁺CCR7⁺CD45RO⁻CD45RA⁺, respectively, and are also identified herein as CCR7⁺CD45RO⁻CD45RA⁺CD4⁺ cells, or CCR7⁺CD45RO⁻CD45RA⁺CD8⁺ cells, respectively. In some embodiments, the naïve T cells are naïve CD4⁺ cells. In some embodiments, the naïve T cells are naive CD8⁺ cells.

Central memory T cells (T_{CM}) are identified by the markers: CD45+CD3+CD4/CD8⁺ CCR7+CD45RA⁻ (or CCR7+CD45RA⁻CD4⁺ or CD8⁺ cells).

In some embodiments, the method further comprises measuring in a blood sample obtained from the subject or a fraction thereof the level of central memory T cells. In some embodiments, the central memory T cells are central memory CD4⁺ cells. In some embodiments, the central memory T cells are central memory CD8⁺ T cells.

Effector memory T cells (T_{EM}) are identified by the markers: CD45⁺CD3⁺ CD4/CD8⁺ CCR7⁻CD45RA⁻ (or CCR7⁻CD45RA⁻CD4⁺ or CD8⁺ cells).

In some embodiments, the method further comprises measuring in a blood sample obtained from the subject or a fraction thereof the level of effector memory T cells. In some embodiments, the effector memory T cells are effector memory CD4⁺ cells. In some embodiments, the effector memory T cells are Effector memory CD8⁺ T cells.

Terminally differentiated effector memory T cells (T_{EMRA}) are identified by the markers: CD45⁺CD3⁺CD4/CD8⁺ CCR7⁻CD45RA⁺ (or CCR7⁻CD45RA⁺CD4⁺ or CD8⁺ cells).

In some embodiments, the method further comprises measuring in a blood sample obtained from the subject or a fraction thereof the level of terminally differentiated effector memory T cells. In some embodiments, the terminally differentiated effector memory T cells are terminally differentiated Effector memory CD4+ cells. In some embodiments, the terminally differentiated effector memory T cells are terminally differentiated effector memory CD8⁺T cells.

HLA-DR T cells are CD4⁺ or CD8⁺ T cells identified by the markers: CD45⁺CD3⁺CD4/CD8⁺ HLA-DR⁺ (or HLA-DR⁺CD4⁺ or CD8⁺ cells). In some embodiments, the HLA-DR T cells are CD4⁺ HLA-DR⁺ T cells. In some embodiments, the HLA-DR T cells are CD8⁺ HLA-DR⁺ T cells.

Activated (or effector) CD4⁺ T cells are identified by the markers: CD45⁺CD3⁺CD4⁺CCR7⁻CD45RO⁻CD45RA⁺, and are also identified herein as CCR7⁻CD45RO⁻ CD45RA⁺ CD4⁺ cells. Effector CD8⁺ T cells are identified by the markers: CD45⁺CD3⁺CD8⁺CCR7⁻CD45RO⁻CD45RA⁺, and are also identified herein as CCR7⁻CD45RO⁻CD45RA⁺CD8⁺ cells.

Classical monocytes are identified by the markers: CD45⁺CD11c⁺ CD14⁺ CD16⁻.

In some embodiments, the method further comprises measuring in a blood sample obtained from the subject or a fraction thereof the level of classical monocytes.

Plasma cells are identified by the markers: CD45⁺CD19⁺CD27⁺.

In some embodiments, the method further comprises measuring in a blood sample obtained from the subject or a fraction thereof the level of plasma cells.

Interferon gamma (IFNγ)-producing T cells are identified by the markers: CD45⁺CD3⁺ and IFNγ⁺.

In some embodiments, the method further comprises measuring in a blood sample obtained from the subject or a fraction thereof the level of IL-22.

In some embodiments, the method further comprises measuring in a blood sample obtained from the subject or a fraction thereof the level of IL-4.

In some embodiments, the method further comprises measuring in a blood sample obtained from the subject or a fraction thereof the level of IL-10.

In all the above definition and embodiments relating to the cell identity by cellular markers, the cells may further be identified by any or all of the following negative markers: CD66b⁻, CD14−, CD20−, and TCRγδ⁻.

The level of many of the cell types is presented as a relative value, a percentage out of the total level of a more generic cell type (usually CD4⁺ or CD8⁺ cells). For example, when the CD4⁺ CD38⁺ T cell level is measured as a percentage out of the total CD4⁺ cells, the term “total” relates to the total level of the more generic cell type in the same sample.

Measuring the level of the different cell types may be carried out by any method known in the art, for example methods based on flow-cytometry, such as Fluorescence-activated cell sorting (FACS), and methods based on mass cytometry such as Cytometry by time of flight (CyTOF).

The term “likely developing” as used herein means that although the subject may still not be presenting with symptoms of the neurodegenerative disease, disorder, or condition, he is probably already developing the disease and will present with symptoms in the future.

The term “measuring”, as used herein e.g. with reference to “at least one biomarker”, means assessing the quantity of the at least one biomarker, including any calculation needed. For example, the measuring may be done by a direct measurement of the concentration or quantity of a certain parameter or biomarker in blood or fraction thereof. Alternatively, the measuring may be done by measuring additional parameters (such as an additional cell type) and calculating, for example, the ratio between the biomarker and the additional parameters.

The term “increased level” as used herein means an increased measurement of the biomarker or other parameter compared to the respective reference (as defined below).

The term “decreased level” as used herein means a decreased measurement of the biomarker or other parameter compared to the respective reference (as defined below).

The increased level or decreased level may be increased or decreased by at least 10%, at least 20%, at least 30%, at least 40%, at least 50%, at least 60%, at least 70%, at least 80%, or at least 90% relative to the respective reference.

The term “respective reference” as used herein corresponds to the normal level, in blood or in plasma or serum, of the respective biomarker. The respective reference may also be a threshold derived from the normal level of the respective biomarker, by additional calculations. The respective reference may be a pre-determined number or range obtained based on prior or ad-hoc research or study, public knowledge in the field, or from measuring the respective biomarker in a collection of individuals not at risk for, or not suspected of having, the respective neurodegenerative disease, disorder, or condition. For example, the respective reference may be an average level of the respective biomarker or parameter measured, in blood or in plasma or serum of: a healthy population; a population not diagnosed for the respective neurodegenerative disease, disorder, or condition; a population not at risk for having the respective neurodegenerative disease, disorder, or condition; a population not carrying the same genetic abnormality as the subject at risk of developing or suspected of having the neurodegenerative disease, disorder, or condition; or a combination thereof. Alternatively, the respective reference may be the published average, minimum, or maximum normal level of the respective biomarker, in blood or in plasma or serum.

In some embodiments, the respective reference is the level of the respective biomarker in healthy individuals. In some embodiments, the respective reference is the level of the respective biomarker in individuals not suffering from, and/or not at risk for, having the neurodegenerative disease, disorder, or condition. In some embodiments, the respective reference is a predetermined value representing the normal levels of the respective biomarker.

The levels of the respective reference are measured in the same type of sample that the respective biomarkers levels are measured, e.g. in blood or in plasma or serum.

The term “blood sample or a fraction thereof” as used herein relates to a blood sample, or a fraction of blood, such as plasma or serum.

CD38 (cluster of differentiation 38), also known as cyclic ADP ribose hydrolase, is a glycoprotein found on the surface of many immune cells, including CD4, CD8, B lymphocytes, and natural killer cells. CD38 levels on regulatory CD4⁺ or CD8⁺ T cells (Tregs) correlate with their suppressive function (Camacho-Pereira et al., 2016).

The inventors have found increased levels of CD38⁺ T cells out of total CD4⁺ or CD8⁺ cells in samples obtained from APP gene duplication carriers compared to samples obtained from control individuals who were not carrying the APP gene duplication (FIG. 2A, B). More specifically, the level of CD38^{hi} T cells was elevated in pre-symptomatic individuals. While the average level of CD38 out of CD4⁺ cells was about 35% in the controls (which in this case were the “respective reference”), it was about 49% in pre-symptomatic AD subjects (an increase of about 40%). Further, while the average levels of CD38 out of CD8⁺ cells was about 17% in samples obtained from the controls (the respective reference), it was about 32% in pre-symptomatic AD subjects (an increase of about 90%).

In some embodiments, the at least one biomarker comprises or consists of CD38⁺ PBMCs. In some embodiments, the at least one biomarker comprises or consists of CD38⁺ T cells. In some embodiments, the at least one biomarker comprises or consists of CD38⁺ CD4⁺ T cells. In some embodiments, the at least one biomarker comprises or consists of CD38⁺ CD8⁺ T cells.

In some embodiments, the at least one biomarker comprises or consists of CD38⁺ CD4⁺ T cells and CD38⁺ CD8⁺ T cells.

In some embodiments, the at least one biomarker comprises or consists of CD38⁺ CD4⁺ T cells, CD38⁺ CD8⁺ T cells, and CD38⁺ classical monocytes.

In some embodiments, the at least one biomarker comprises or consists of CD38⁺ CD4⁺ T cells, CD38⁺ CD8⁺ T cells, and CD38⁺ plasma cells or B cells.

In some embodiments, the at least one biomarker comprises or consists of CD38⁺ CD4⁺ T cells, CD38⁺ CD8⁺ T cells, CD38⁺ classical monocytes, and CD38⁺ plasma cells or B cells.

In some embodiments, the at least one biomarker comprises or consists of CD38⁺ CD4⁺ T cells, CD38⁺ CD8⁺ T cells, and CD4⁺ CD45RA T cells T cells, and/or CD8⁺ CD45RA⁺ T cells.

In some embodiments, the increased CD38⁺ PBMCs level compared to the respective reference for CD38⁺ PBMCs level is increased by at least about 10%, 15%, 20%, 25%, 30%, 40%, 50%, 70%, 90%, or 100%.

In some embodiments, the increased CD38⁺ T cells level compared to the respective reference for CD38⁺ T cells level is increased by at least about 10%, 15%, 20%, 25%, 30%, 40%, 50%, 70%, 90%, or 100%.

In some embodiments, the CD38⁺ PBMCs level is calculated by measuring the levels of (1) CD38⁺ PBMCs and (2) total PBMCs in a sample, and calculating the proportion of (1) out of (2).

In some embodiments, the at least one biomarker comprises CD38⁺ CD4⁺ T cells.

In some embodiments, the at least one biomarker comprises CD38⁺ CD8⁺ T cells.

In some embodiments, the at least one biomarker comprises CD38⁺ CD4⁺ T cells and CD38⁺ CD8⁺ T cells.

In some embodiments, the CD38⁺ CD4⁺ T cells level is calculated by measuring the levels of (1) CD38⁺ CD4⁺ cells and (2) total CD4⁺ cells in a sample, and calculating the proportion of (1) out of (2).

In some embodiments, the CD38⁺ CD8⁺ T cells level is calculated by measuring the levels of (1) CD38⁺ CD8⁺ cells and (2) total CD8⁺ cells, and calculating the proportion of (1) out of (2).

In some embodiments, the CD38⁺ T cells level is calculated by measuring the levels of (1) CD38⁺ CD4⁺ and CD38⁺ CD8⁺ cells and (2) total CD4⁺ and CD8⁺ cells, and calculating the proportion of (1) out of (2).

In some embodiments, the at least one biomarker comprises CD38⁺ PBMCs level calculated by measuring the levels of (1) CD38⁺ PBMCs and (2) total PBMCs, and calculating the proportion of (1) out of (2), and the increased CD38⁺ PBMCs level compared to the respective reference for CD38⁺ PBMCs level is increased by at least about 10%, 15%, 20%, 25%, 30%, 40%, 50%, 60%, 70%, 80%, 90%, or 100%.

In some embodiments, the at least one biomarker comprises CD38⁺ T cells level calculated by measuring the levels of (1) CD38⁺ CD4⁺ cells and (2) total CD4⁺ cells, and calculating the proportion of (1) out of (2), and the increased CD38⁺ T cells level compared to the respective reference for CD38⁺ T cells level is increased by at least about 10%, 15%, 20%, 25%, 30%, 40%, 50%, 60%, 70%, 80%, 90%, or 100%.

In some embodiments, the at least one biomarker comprises CD38⁺ T cells level calculated by measuring the levels of (1) CD38⁺ CD8⁺ cells and (2) total CD8⁺ cells, and calculating the proportion of (1) out of (2), and the increased CD38⁺ T cells level compared to the respective reference for CD38⁺ T cells level is increased by at least about 10%, 15%, 20%, 25%, 30%, 40%, 50%, 60%, 70%, 80%, 90%, 100%, 120%, 140%, 150%, 160%, 180%, or 200%.

In some embodiments, the at least one biomarker comprises CD38⁺ T cells level calculated by measuring the levels of (1) CD38⁺ CD4⁺ cells and (2) total CD4⁺ cells, and calculating the proportion of (1) out of (2), and the respective reference for CD38⁺ T cells level is between about 35% and about 45%, or between about 35% and about 40%, CD38⁺ CD4⁺ cells out of total CD4⁺ cells.

In some embodiments, the respective reference for CD38⁺ T cells level is at least about 36%, 38%, or 40% CD38⁺ CD4⁺ cells out of total CD4⁺ cells.

In some embodiments, the at least one biomarker comprises CD38⁺ T cells level calculated by measuring the levels of (1) CD38⁺ CD8⁺ cells and (2) total CD8⁺ cells, and calculating the proportion of (1) out of (2), and the respective reference for CD38⁺ T cells level is between about 15% and about 30%, or between about 15% and about 25%, CD38⁺ CD8⁺ cells out of total CD8⁺ cells.

In some embodiments, the respective reference for CD38⁺ T cells level is at least about 18%, 20%, or 25% CD38⁺ CD8⁺ cells out of total CD8⁺ cells.

In some embodiments, the CD38⁺ T cells are CD38^{hi} T cells. In some embodiments, the CD38⁺ T cells are CD38^{h} CD4⁺ T cells. In some embodiments, the CD38⁺ T cells are CD38^{hi} CD8⁺ T cells.

In some embodiments, the CD38⁺ T cells are CD38^{lo} T cells. In some embodiments, the CD38⁺ T cells are CD38^{lo} CD4⁺ T cells. In some embodiments, the CD38⁺ T cells are CD38^{lo} CD8⁺ T cells.

The inventors have additionally found an increased Tₕ2 population level, a decreased Tₕ1 population level, and a resulting increased Tₕ2/Tₕ1 ratio in pre-symptomatic APP gene duplication carriers compared to control individuals who were not carrying the APP gene duplication (FIG. 1B). While the average Tₕ2/Tₕ1 ratio was about 9 in the controls, it was about 15 AD pre-symptomatic carriers, i.e., an increase of about 66%.

In some embodiments, the at least one biomarker comprises or consists of Tₕ2 cells.

In some embodiments, the at least one biomarker comprises or consists of Tₕ2/Tₕ1 ratio.

In some embodiments, the level of the Tₕ2 cells is measured by measuring the level of CXCR5⁻CCR6⁻CXCR3⁻CD4⁺ cells and/or at least IL-4 producing CD4⁺ T cells.

In some embodiments, the Tₕ2/Tₕ1 ratio is calculated by measuring the level of (1) CXCR5⁻CCR6⁻CXCR3⁻CD4⁺ cells (Tₕ2), and (2) CXCR5⁻CCR6⁻CXCR3⁺CD4⁺ cells (Tₕ1), and calculating the proportion of (1) out of (2).

In some embodiments, the increased Tₕ2/Tₕ1 ratio compared to the respective reference is increased by at least 10%, 20%, 30%, 35%, 40%, 45%, 50%, 60%, or 70%, 80%, 90%, 100%, 120%, 140%, 150%, 160%, 180%, or 200%. In some embodiments, the increased Tₕ2/Tₕ1 ratio compared to the respective reference is increased by at least 30%, at least 40%, at least 50% or at least 60%, or at least 70% compared to the respective reference.

In some embodiments, the respective reference for Tₕ2/Tₕ1 ratio is about 8, 9, 10, 11, 12, 13, or 14.

In some embodiments, the Tₕ2/Tₕ1 ratio is determined by the level of IL-4 producing CD4⁺ T cells. Accordingly, an increase in the Tₕ2/Tₕ1 ratio is defined by an increase in the levels of IL-4 producing CD4⁺ T cells. In some embodiments, the Tₕ2 response can be determined by the level of IL-4 producing CD4⁺ T cells. Accordingly, an increase in the Tₕ2 response is defined by an increase in the levels of IL-4 producing CD4⁺ T cells.

The inventors have further found an increased naïve CD4⁺ T cells level in APP gene duplication carriers compared to control individuals who were not carrying the APP gene duplication (FIG. 1D). While the average naïve CD4⁺ T cells level out of the total CD4⁺ T cells was about 35% in the controls, it was about 50% in the AD pre-symptomatic subjects, i.e., an increase of about 42%.

In some embodiments, the at least one biomarker comprises or consists of naïve T cells level. In some embodiments, the at least one biomarker comprises or consists of naïve CD4⁺ T cells level. In some embodiments, the at least one biomarker comprises or consists of naïve CD8⁺ T cells level.

In some embodiments, the naïve CD4⁺ T cells level is calculated by measuring the level of (1) CCR7⁺CD45RO⁻CD45RA⁺ (naïve) CD4⁺ cells, and (2) total CD4⁺ cells, and calculating the proportion or frequency of (1) out of (2).

In some embodiments, the increased naïve CD4⁺ T cells level compared to the respective reference is increased by at least 30%, 35%, 40%, 45%, 50%, 55%, 60%, 65%, 70%, 75%, 80%, 85%, 90%, 95%, or 100%.

In some embodiments, the respective reference for naïve CD4⁺ T cells level is at least 15%, 20%, 25%, or 30% out of the total CD4⁺ cells.

In some embodiments, the method further comprises measuring in the sample naïve CD8⁺ T cells level, wherein an increased level of naïve CD8⁺ T cells as compared to a respective reference, indicates that the subject is likely developing, or affected by, said neurodegenerative disease, disorder, or condition.

In some embodiments, the naïve CD8⁺ T cells level is calculated by measuring the level of (1) CCR7⁺CD45RO⁻CD45RA⁺ (naïve) CD8⁺ cells, and (2) total CD8⁺ cells, and calculating the proportion of (1) out of (2).

The inventors have found a decreased effector memory CD8⁺ T cells level in APP gene duplication carriers compared to control individuals who were not carrying the APP gene duplication (FIG. 1E)). While the average effector CD8⁺ T cells level out of the total CD8⁺ T cells was about 17% in the controls, it was about 12% in the AD pre-symptomatic subjects, i.e., a decrease of about 83%.

In some embodiments, the method further comprises measuring in the sample effector CD8⁺ T cells level, wherein a decreased level of effector CD8⁺ T cells as compared to a respective reference, indicates that the subject is likely developing, or affected by, said neurodegenerative disease, disorder, or condition.

In some embodiments, the effector CD8⁺ T cells level is calculated by measuring the level of (1) CCR7⁻CD45RO⁻CD45RA⁺ (effector) CD8⁺ cells, and (2) total CD8⁺ cells, and calculating the proportion of (1) out of (2).

In some embodiments, the decreased effector CD8⁺ T cells level compared to the respective reference is decreased by at least about 20%, 25%, 30%, 35%, 40%, 45%, or 50%.

In some embodiments, the respective reference for effector CD8⁺ T cells level is at most about 20%, 19%, or 18%, 17%, 16%, or 15% out of the total CD8⁺ cells.

The inventors further found a decrease in activated CD4⁺ T cells level out of the total CD4⁺ T cells (1.3% in healthy controls, 0.7% in pre-symptomatic, and 0.5% in symptomatic AD—a decrease of about 40-60%, data not shown).

In some embodiments, the at least one biomarker comprises or consists of activated CD4⁺ T cells level.

In some embodiments, the activated CD4⁺ T cells level is calculated by measuring the level of (1) CCR7⁻CD45RO⁻CD45RA⁺ (effector) CD4⁺ cells, and (2) total CD4⁺ cells, and calculating the proportion of (1) out of (2).

In some embodiments, the decreased effector CD4⁺ T cells level compared to the respective reference is decreased by at least about 20%, 25%, 30%, 35%, 40%, 45%, 50%, 55%, 60%, 65%, 70%, 75%, or 80%.

CD38 is known to be associated with mitochondrial metabolic dysfunction. In view of the foregoing, the inventors tested levels of metabolites in plasma, and found a decreased level of plasma metabolites in APP gene duplication carriers compared to control individuals who were not carrying the APP gene duplication. The metabolites for which the earliest reduction was found are the following: trigonelline of the nicotinate and nicotinamide metabolism (FIGS. 4C and 4E), allose of the fructose/mannose metabolism (FIG. 4B), and 3-(3-hydroxyphenyl)propionic acid, of the phenylalanine metabolism (data not shown).

Further, the inventors have found reduced levels of the glucose transporter 1 (GLUT1) in CD4⁺ cells (FIG. 3C).

In some embodiments, the at least one biomarker comprises or consists of the level of GLUT1 in CD4⁺ cells.

In some embodiments, the level of GLUT1 in CD4⁺ cells is reduced by at least 10%, at least 20%, at least 30%, at least 40%, at least 50%, at least 60%, or at least 70% relative to the respective reference.

In some embodiments, the level of GLUT1 in CD4⁺ cells is the level of GLUT1 expressed on the surface of CD4⁺ cells.

In addition, the following metabolites were somewhat decreased in pre-symptomatic individuals compared to controls: citrate, 2, 6-dihydroxybenzoic acid; hypotaurine; and indoleacetaldehyde.

It should be pointed out that the development of AD is a long process which takes years and is sped up in patients with a genetic predisposition, such as the patients having the APP gene duplication of the study described here. Therefore, pre-symptomatic carriers of the APP duplication include carriers who are far along this process and are very close to developing AD symptoms, as well as carriers who are in the beginning of the process and are far from developing full-blown symptoms. Accordingly, any of these deviations from the normal levels are indicative of a neurodegenerative process.

In view of the foregoing, the different metabolites level may be indicative of the stage in the process that the individual is in. For example, trigonelline, allose, and 3-(3-hydroxyphenyl)propionic acid may be considered as earlier biomarkers, the levels of which are decreased early in the process. As a result, a clear decrease can already be observed in the group of pre-symptomatic carriers. On the other hand, choline glycerophosphate, creatine, N-acetylneuraminate, and 2-propylpentanoic acid may be considered as late markers, in which changes in levels compared to controls are observed rather late in the process, possibly very close to appearance of cognitive symptoms. Accordingly, only the symptomatic patients demonstrate a change in these metabolites compared to controls. In between the two extremes are citrate, 2, 6-dihydroxybenzoic acid; hypotaurine; and indoleacetaldehyde, which start the decrease at some point during the process and keep decreasing until cognitive symptoms appear. For these metabolites, some change is already observed in pre-symptomatic carriers, but a greater change is seen in symptomatic patients.

In some embodiments, the at least one biomarker comprises or consists of plasma or serum levels of at least one metabolite of a pathway selected from nicotinate and nicotinamide metabolism, fructose/mannose metabolism, and phenylalanine metabolism.

In some embodiments, the at least one biomarker comprises or consists of plasma or serum levels of at least one metabolite selected from trigonelline, allose, and 3-(3-hydroxyphenyl)propionic acid.

In some embodiments, the at least one biomarker comprises or consists of plasma or serum levels of at least one metabolite selected from trigonelline, allose, and adenosine.

In some embodiments, the at least one biomarker comprises or consists of plasma or serum levels of at least one metabolite selected from trigonelline and allose.

In some embodiments, the at least one biomarker comprises or consists of plasma or serum levels of trigonelline. In some embodiments, the at least one biomarker comprises or consists of plasma or serum levels of allose. In some embodiments, the at least one biomarker comprises or consists of plasma or serum levels of 3-(3-hydroxyphenyl)propionic acid.

In some embodiments, the method further comprises measuring in plasma or serum the levels of at least one metabolite selected from citrate, 2, 6-dihydroxybenzoic acid; hypotaurine; indoleacetaldehyde; choline glycerophosphate; creatine; N-acetylneuraminate; and 2-propylpentanoic acid; wherein an increased level of at least one of creatine; N-acetylneuraminate; and 2-propylpentanoic acid as compared to a respective reference, and/or a decreased level of at least one of choline glycerophosphate; citrate; 2, 6-dihydroxybenzoic acid; hypotaurine; and indoleacetaldehyde as compared to a respective reference, indicates that the subject is likely developing, or affected by, said neurodegenerative disease, disorder, or condition.

In some embodiments, the decreased plasma or serum level of the at least one metabolite compared to the respective reference is decreased by at least about 10%, 20%, 30%, 40%, 50%, 60%, 70%, 80%, or 90%.

In some embodiments, the increased plasma or serum level of the at least one metabolite compared to the respective reference is increased by at least about 10%, 20%, 30%, 40%, 50%, 60%, 70%, 80%, or 90%.

As seen in FIG. 4G, the level of adenosine is increased in pre-symptomatic patients.

Accordingly, in some embodiments, the at least one biomarker comprises or consists of plasma or serum levels of adenosine.

In some embodiments, the increased plasma or serum level of the adenosine compared to the respective reference is increased by at least about 10%, 20%, 30%, 40%, 50%, 60%, 70%, 80%, or 90%.

The pre-symptomatic APP duplication carriers did not show an increased level of neurofilament light chain, as did the symptomatic AD patients (FIG. 1F). This indicates that neuronal degeneration has not yet started in these individuals.

Accordingly, in some embodiments, the level of plasma neurofilament light chain (NfL) in the subject is not increased compared to the NfL levels in the respective reference. In some embodiments, the level of plasma neurofilament light chain (NfL) in the subject is not increased above about 15 pg/ml, about 20 pg/ml, about 25 pg/ml, about 30 pg/ml, about 35 pg/ml, about 40 pg/ml, or about 45 pg/ml. In some embodiments, the level of plasma neurofilament light chain (NfL) in the subject is not increased above 20 pg/ml.

Using machine learning tools, the inventors discovered that certain combinations of biomarkers provide a better separation between presymptomatic and healthy individuals. Three combinations were found to be especially valuable: 1) CD38+CD4 and CD8 T cells; 2) CD38+CD4 and CD8 T cells with naïve CD4 and CD8 cells; and 3) CD38+CD4 T cells, CD8 T cells, classical monocytes, and plasma cells), see Example 4.

In some embodiments, the at least one biomarker comprises CD38⁺ CD4⁺ T cells. In some embodiments, the at least one biomarker comprises CD38⁺ CD8⁺ T cells. In some embodiments, the at least one biomarker comprises CD38⁺ CD4⁺ T cells and CD38⁺ CD8⁺ T cells.

In some embodiments, the at least one biomarker comprises CD38⁺ CD4⁺ T cells, CD38⁺ CD8⁺ T cells, naïve CD4⁺ T cells, and naïve CD8⁺ T cells.

In some embodiments, the at least one biomarker comprises CD38⁺ CD4⁺ T cells, CD38⁺ CD8⁺ T cells, CD38⁺ classical monocytes, and CD38⁺ plasma (B) cells.

In some embodiments, the at least one biomarker comprises CD38⁺ PBMCs and Tₕ2 cells. In some embodiments, the at least one biomarker comprises CD38⁺ PBMCs and Tₕ2/Tₕ1 ratio.

In some embodiments, the at least one biomarker comprises CD38⁺ T cells and Tₕ2 cells. In some embodiments, the at least one biomarker comprises CD38⁺ T cells and Tₕ2/Tₕ ratio.

In some embodiments, the at least one biomarker comprises CD38^{hi} I cells and Tₕ2 cells. In some embodiments, the at least one biomarker comprises CD38^{hi} T cells and Tₕ2/Tₕ1 ratio.

In some embodiments, the at least one biomarker comprises CD38⁺ PBMCs, and at least one biomarker selected from Tₕ2/Tₕ1 ratio, naïve CD4⁺ T cells level and activated CD4⁺ T cells level.

In some embodiments, the at least one biomarker comprises CD38⁺ T cells, and at least one biomarker selected from Tₕ2/Tₕ1 ratio, naïve CD4⁺ T cells level and activated CD4⁺ T cells level.

In some embodiments, the at least one biomarker comprises CD38⁺ PBMCs, Tₕ2/Tₕ1 ratio, and at least one biomarker selected from naïve CD4⁺ T cells and activated CD4⁺ T cells.

In some embodiments, the at least one biomarker comprises CD38⁺ T cells, Tₕ2/Tₕ1 ratio, and at least one biomarker selected from naïve CD4⁺ T cells and activated CD4⁺ T cells.

In some embodiments, the at least one biomarker comprises or consists of CD38⁺ CD4⁺ T cells, Tₕ2 cells, and CD4⁺ naïve T cells. In some embodiments, the at least one biomarker comprises or consists of CD38^{hi} CD4⁺ T cells, Tₕ2 cells, and CD4⁺ naïve T cells.

In some embodiments, the at least one biomarker comprises CD38⁺ PBMCs and at least one metabolite selected from trigonelline, allose, and adenosine.

In some embodiments, the at least one biomarker comprises CD38⁺ T cells and at least one metabolite selected from trigonelline, allose, and adenosine.

In some embodiments, the at least one biomarker comprises CD38⁺ PBMCs and trigonelline.

In some embodiments, the at least one biomarker comprises CD38⁺ T cells and trigonelline.

In some embodiments, the at least one biomarker comprises CD38⁺ CD4⁺ T cells and trigonelline.

In some embodiments, the at least one biomarker comprises CD38^{hi} T cells and at least one metabolite selected from trigonelline, allose, and adenosine.

In some embodiments, the at least one biomarker comprises CD38^{hi} T cells and trigonelline.

In some embodiments, the at least one biomarker comprises CD38^{hi} CD4⁺ T cells and trigonelline.

In some embodiments, increased levels of CD38+ PBMCs compared to a respective reference and decreased levels of trigonelline as compared to a respective reference indicate that the subject is likely developing, or affected by, said neurodegenerative disease, disorder, or condition.

In some embodiments, increased levels of CD38+ T cells compared to a respective reference and decreased levels of trigonelline as compared to a respective reference indicate that the subject is likely developing, or affected by, said neurodegenerative disease, disorder, or condition.

In some embodiments, increased levels of CD38+ PBMCs compared to a respective reference and decreased levels of GLUT1 expression in CD4⁺ T cells as compared to a respective reference indicate that the subject is likely developing, or affected by, said neurodegenerative disease, disorder, or condition

In some embodiments, increased levels of CD38+ T cells compared to a respective reference and decreased levels of GLUT1 expression in CD4⁺ T cells as compared to a respective reference indicate that the subject is likely developing, or affected by, said neurodegenerative disease, disorder, or condition

In some embodiments, the at least one biomarker comprises CD38⁺ PBMCs, Tₕ2 or Tₕ2/Tₕ1 ratio, and at least one metabolite selected from trigonelline, allose, and adenosine.

In some embodiments, the at least one biomarker comprises CD38⁺ T cells, Tₕ2 or Tₕ2/Tₕ1 ratio, and at least one metabolite selected from trigonelline, allose, and adenosine.

In some embodiments, the at least one biomarker comprises CD38^{hi} T cells, Tₕ2 or Tₕ2/Tₕ1 ratio, and at least one metabolite selected from trigonelline, allose, and adenosine.

In some embodiments, the at least one biomarker comprises Tₕ2 or Tₕ2/Tₕ1 ratio, and the at least one metabolite selected from trigonelline, allose, and adenosine.

The term “a neurodegenerative disease, disorder, or condition” as used herein relates to a disease, disorder or condition which affects brain function. Non-limiting examples of a neurodegenerative disease, disorder, or condition are: AD, amyotrophic lateral sclerosis (ALS), Parkinson's disease, Huntington's disease, primary progressive multiple sclerosis; secondary progressive multiple sclerosis, attention deficit disorder (ADD), corticobasal degeneration, Creutzfeldt-Jakob disease (CJD), Rett syndrome, a retinal degeneration disorder selected from the group consisting of age-related macular degeneration and retinitis pigmentosa; anterior ischemic optic neuropathy; glaucoma; uveitis; depression; trauma-associated stress or post-traumatic stress disorder, frontotemporal dementia (FTD), Lewy body dementias, mild cognitive impairments, posterior cortical atrophy, primary progressive aphasia or progressive supranuclear palsy.

This term further refers to Tauopathies, a clinically, morphologically and biochemically heterogeneous class of neurodegenerative diseases characterized by a pathological aggregation of tau protein in neurofibrillary or gliofibrillary tangles in the human brain. Tau is a microtubule-associated protein (MAP) that binds to microtubules and promotes their polymerization. It plays an important role in maintaining axonal transport and neuronal integrity but has a physiological role in dendrites, and it is expressed at low levels in glial cells. In a tauopathy, tangles are formed by hyperphosphorylation of tau causing it to aggregate in an insoluble form. Non-limiting examples of tauopathies include Alzheimer's disease, argyrophilic grain disease, chronic traumatic encephalopathy, corticobasal degeneration, dementia pugilistica, frontotemporal dementia, frontotemporal lobar degeneration, Hallervorden-Spatz disease, Huntington's disease, ganglioglioma, gangliocytoma, globular glial tauopathy, lead encephalopathy, lipofuscinosis, Lytico-Bodig disease (Parkinson-dementia complex of Guam), meningioangiomatosis, Parkinsonism disease linked to chromosome 17, Pick's disease, primary age-related tauopathy (PART), formerly known as neurofibrillary tangle-only dementia (NFT-dementia), postencephalitic parkinsonism, progressive supranuclear palsy, subacute sclerosing panencephalitis and tuberous sclerosis.

The term further refers to a neurodegenerative process resulting from an injury of the CNS, such as spinal cord injury, closed head injury, blunt trauma, penetrating trauma, hemorrhagic stroke, ischemic stroke, cerebral ischemia, optic nerve injury, myocardial infarction, organophosphate poisoning and injury caused by tumor excision.

In some embodiments, related to any of the above embodiments, the neurodegenerative disease, disorder or condition is selected from AD, amyotrophic lateral sclerosis (ALS), Parkinson's disease, Huntington's disease, primary progressive multiple sclerosis; secondary progressive multiple sclerosis, attention deficit disorder (ADD), corticobasal degeneration, Creutzfeldt-Jakob disease (CJD), Rett syndrome, a retinal degeneration disorder selected from the group consisting of age-related macular degeneration and retinitis pigmentosa; anterior ischemic optic neuropathy; glaucoma; uveitis; depression; trauma-associated stress or post-traumatic stress disorder, frontotemporal dementia (FTD), Lewy body dementias, mild cognitive impairments, posterior cortical atrophy, primary progressive aphasia or progressive supranuclear palsy, or any of the tauopathies CNS injuries mentioned above.

In some embodiments, related to any of the above embodiments, the neurodegenerative disease, disorder or condition is AD.

The present invention additionally provides, in specific embodiments, a method for early detection or diagnosis of Alzheimer's disease (AD) in a subject at risk of developing or suspected of having AD, the method comprising measuring in a blood sample obtained from the subject the levels of at least one biomarker selected from CD38⁺ peripheral blood mononuclear cells (PBMCs), trigonelline, GLUT1 expression in CD4⁺ T cells, Tₕ2, Tₕ2/Tₕ1 ratio, naïve T cells, adenosine, allose, and HLA-DR T cells,

wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicate that the subject is likely developing, or affected by, AD.

In another aspect, the present invention provides a method for early detection or diagnosis of Alzheimer's disease (AD) in a subject at risk of developing or suspected of having AD, the method comprising measuring in a blood sample obtained from the subject or a fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells,

wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicate that the subject is likely developing, or affected by, AD.

In another aspect, the present invention provides a method for stratifying a subject in a subgroup of a clinical trial of an IFN-γ dependent immune checkpoint modulator for the treatment of a neurodegenerative disease, disorder, or condition, the method comprising measuring in a blood sample obtained from the subject or a fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells,
wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that that the subject is likely to be responsive to treatment with the immune checkpoint modulator, and
no change or a decreased level of at least one of CD38⁺ PBMCs, Tₕ2, Tₕ2/Tₕ1 ratio, naïve T cells, and adenosine, as compared to a respective reference, and/or no change or an increased level of at least one of trigonelline, GLUT1 expression in CD4⁺ T cells, allose, and HLA-DR T cells as compared to a respective reference, indicates that that the subject is likely not to be responsive to treatment with the immune checkpoint modulator, and said biomarker enables the stratification of said subject into a subgroup for said clinical trial.

In a further aspect, the present invention provides a method for predicting whether a patient diagnosed with a neurodegenerative disease, disorder, or condition is likely to be responsive or non-responsive to treatment with an IFN-γ dependent inhibitory immune checkpoint blockade/modulator, said method comprising measuring in a blood sample obtained from the subject or a fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells,
wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that the patients is likely to be responsive to treatment with said immune checkpoint modulator (predisposition to responsiveness), and
no change or a decreased level of at least one of CD38⁺ PBMCs, Tₕ2, Tₕ2/Tₕ1 ratio, naïve T cells, and adenosine, as compared to a respective reference, and/or no change or an increased level of at least one of trigonelline, GLUT1 expression in CD4⁺ T cells, allose, and HLA-DR T cells as compared to a respective reference, indicates that the patients is likely to be non-responsive to treatment with said immune checkpoint modulator (predisposition to non-responsiveness).

In certain embodiments, said patient is further diagnosed with reduction in cognitive function prior to said treatment, and said indication that the patient is likely to be responsive predicts an improvement in cognitive function.

In certain embodiments, in case the patient is likely to be responsive, said treatment is initiated or continued; and in case the patient is likely to be non-responsive, said treatment is not initiated or discontinued.

In still another aspect, the present invention provides a method for excluding a patient diagnosed with a neurodegenerative disease, disorder, or condition from treatment with an IFN-γ dependent immune checkpoint modulator, said method comprising measuring in a blood sample obtained from the subject or a fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells,
wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that the patients is likely to be responsive to treatment with said immune checkpoint modulator (predisposition to responsiveness), and
no change or a decreased level of at least one of CD38⁺ PBMCs, Tₕ2, Tₕ2/Tₕ1 ratio, naïve T cells, and adenosine, as compared to a respective reference, and/or no change or an increased level of at least one of trigonelline, GLUT1 expression in CD4⁺ T cells, allose, and HLA-DR T cells as compared to a respective reference, indicates that the patients is likely to be non-responsive to treatment with said immune checkpoint modulator (predisposition to non-responsiveness) and is therefore excluded from treatment with said immune checkpoint modulator.

In yet another aspect, the present invention provides method of assessing efficacy of a treatment by an IFN-γ dependent immune checkpoint modulator in a subject diagnosed with a neurodegenerative disease, disorder, or condition, said method comprising:
obtaining a first blood sample from the subject;
administering the immune checkpoint modulator to the subject;
obtaining a second blood sample from the subject;
measuring in the first blood sample or a fraction thereof and in the second blood sample or fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells, and
comparing the level of the at least one biomarker in the first blood sample with the level of the biomarker in the second blood sample;
wherein a decreased level of at least one of CD38⁺ PBMCs, Tₕ2, Tₕ2/Tₕ1 ratio, naïve T cells, and adenosine, and/or an increased level of at least one of trigonelline, GLUT1 expression in CD4⁺ T cells, allose, and HLA-DR T cells in the second blood sample compared to the first blood sample indicates that the treatment by an immune checkpoint modulator is likely effective, and
an unchanged or increased level of at least one of CD38⁺ PBMCs, Tₕ2, Tₕ2/Tₕ1 ratio, naïve T cells, and adenosine, and/or an unchanged or decreased level of at least one of trigonelline, GLUT1 expression in CD4⁺ T cells, allose, and HLA-DR T cells in the second blood sample compared to the first blood sample indicates that the treatment by an IFN-γ dependent immune checkpoint modulator is likely ineffective.

It is noted that all the aspects of the application, relating to methods for early detection or diagnosis, stratifying a subject in a subgroup, predicting whether a subject is likely to be responsive to treatment, excluding a subject from treatment, assessing the efficacy of treatment or treating a subject, are meant to include all embodiments mentioned above, including, inter alia, embodiments defining the biomarkers measured and the interpretation of an increase or decrease in the levels of the biomarkers.

The scientific basis for treating with an immune checkpoint modulator is as follows:

Immune checkpoints are regulatory pathways for maintaining systemic immune homeostasis and tolerance. Without wishing to be limited to any theory, selective blockade of IFN-γ dependent immune checkpoints reactivates a systemic IFN-γ-dependent cascade of immunological responses suppressed due to pathologic conditions. The increased IFN-γ signaling results in migration of leukocyte across the choroid plexus epithelium into the CNS territory and recruitment of monocyte-derived macrophages and other immunoregulatory cells (T cells) to diseased sites within the brain as explained above. Importantly, this recruitment results in a comprehensive effect on brain function, including reduced of amyloid plaque burden (in experimental models of Alzheimer's disease), restored immunological balance within the brain parenchyma, reduced neuroinflammation, reduced gliosis, reduced synaptic loss, increased hippocampal neurogenesis, increased neuronal protection and enhanced neuronal survival, collectively leading to neuroprotection and/or mitigation of cognitive decline. Thus, blockade of immune checkpoints restores healthy brain-immune dialogue via increased IFN-γ signaling that enables brain maintenance and repair of a pathologic condition.

By using mouse models of Alzheimer's disease (AD), the inventors have previously demonstrated (WO 2015/136541; WO 2018/047178) that a systemic IFN-γ-dependent immune response was evoked by using neutralizing antibodies for programmed cell death protein 1 (PD-1), PD-L1 and T-cell immunoglobulin and mucin-domain containing-3 (TIM-3) and that this IFN-γ-dependent immune response was needed in order to mobilize immune cells to the CNS. When induced in animals with established AD pathology, treatment with these neutralizing antibodies resulted in an immunological response that cleared of cerebral amyloid-b (AD) plaques and improved cognitive performance. Thus, using neutralizing antibodies for three different immune checkpoint members resulted in an IFN-γ-dependent immune response that reversed the disease state.

Generally, the immune response which is mounted following immune checkpoint blockade is largely associated with IFN-γ or T cells which produce IFN-γ. As such, any immune checkpoint member that suppresses an IFN-γ-dependent immune response would be a highly feasible target in a pathologic condition because neutralizing the activity of these immune checkpoint members would alleviate said immunological suppression, resulting in an IFN-γ-dependent immune response.

With this in mind, immune checkpoint molecule mentioned below are also known to suppress an IFN-γ-dependent immune response, and therefore may be used as targets for treating the neurodegenerative disease, disorder, or condition.

Importantly, some immune checkpoint molecules can be considered as “off switches” on the immune response, their blockade activates the immune system, and thus these are referred to as “negative regulators”. Other immune checkpoint molecules can be considered as “on switches” on the immune response, their stimulation activates the immune system, and thus these are referred to as “positive regulators”. Many of these molecules are members of the B7 family, and they act as rheostats that control the threshold for whether a given T-cell receptor (TCR) interaction leads to activation and/or anergy. Targeting either negative regulators or positive regulators checkpoints leads to an IFN-γ-dependent immune response.

In a further aspect, the present invention provides a method for treating or preventing in a subject at risk for or diagnosed with a neurodegenerative disease, disorder, or condition, said method comprising measuring in a blood sample or fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells,
wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that the subjects is likely developing, or is affected by, the neurodegenerative disease, disorder, or condition, and
no change or a decreased level of at least one of CD38⁺ PBMCs, Tₕ2, Tₕ2/Tₕ1 ratio, naïve T cells, and adenosine, as compared to a respective reference, and/or no change or an increased level of at least one of trigonelline, GLUT1 expression in CD4⁺ T cells, allose, and HLA-DR T cells as compared to a respective reference as compared to a respective reference, indicates that
the subject is not likely affected by the neurodegenerative disease, disorder, or condition, and the method further comprising administering to the subject an active agent, such as an immune checkpoint modulator thereby treating the subject that is likely affected by the neurodegenerative disease, disorder, or condition.

In certain embodiments, the administration of the immune checkpoint modulator involves a treatment regimen that is calibrated such that the level of systemic immunosuppression is transiently reduced.

The term “immune checkpoint modulator” as used herein refers to an IFN-γ dependent immune checkpoint modulator, i.e. an immune checkpoint modulator that suppresses an IFN-γ-dependent immune response.

In certain embodiments, the immune checkpoint modulator is an inhibitor of a negative regulator immune checkpoint selected from PD-L1, PD-1, TIM-3, CTLA-4, A2aR, B7-H3, B7-H4, BTLA, IDO, KIR, LAG-3, NOX2 and VISTA. In certain embodiments, the inhibitor is an antagonistic antibody.

In certain embodiments, the immune checkpoint modulator is an activator of a positive regulator immune checkpoint selected from OX40, ICOS, CD27, CD28, CD40, GITR, CD122 and CD137. In certain embodiments, the inhibitor is an agonistic antibody.

Treatment regimens for administration of the immune checkpoint modulator include those disclosed in WO2015136541, WO2017009829, and WO2018047178.

More specifically, such treatment regimens include at least two courses of therapy, each course of therapy comprising in sequence a treatment session followed by an interval session of non-treatment, or a non-treatment session.

The treatment session may include a single administration, or it may include repeated or multiple administrations provided during a certain period of time.

When the treatment session includes repeated administration:
the length of the treatment session may be 1 day to 3 weeks, one day to two weeks, one day to one week, one day to three days, one day to two days, one day, one week to three weeks, or one week to two weeks;
the frequency of repeated administration may be once a day, once every two days, once every three days, or once a week; and
the length of the non-treatment session is longer than the time (the intervening period) between any two consecutive administrations during the treatment session.

The length of the non-treatment session may be one to six months, one to three months, one to two months, one month, three to six weeks, four to six weeks, or six weeks.

Alternatively, the treatment regimen may include single administration or injection of the modulator once every week, two, three, four, five six, seven, or eight weeks, or once every two, three, four, five or six months.

The term “treating” as used herein refers to means of obtaining a desired physiological effect. The effect may be therapeutic in terms of partially or completely curing a disease and/or symptoms attributed to the disease. The term refers to inhibiting the disease, i.e. arresting or slowing its development; or ameliorating the disease, i.e. causing regression of the disease.

It is noted that in the present application, measurements carried out in plasma may also be carried out in serum. Accordingly, any reference to plasma is intended to refer to plasma or serum.

The determination of the doses of the immune checkpoint modulator to be used for human use is based on commonly used practices in the art, and will be finally determined by physicians in clinical trials. An expected approximate equivalent dose for administration to a human can be calculated based on the in vivo experimental evidence disclosed herein below, using known formulas (e.g. Reagan-Show et al. (2007) Dose translation from animal to human studies revisited. The FASEB Journal 22:659-661). According to this paradigm, the adult human equivalent dose (mg/kg body weight) equals a dose given to a mouse (mg/kg body weight) multiplied with 0.081.

In a further aspect, the present invention provides compositions and kits for use in the early detection and diagnosis of a neurodegenerative disease, disorder, or condition in a subject at risk of developing or suspected of having the neurodegenerative disease, disorder, or condition. The kits or compositions include reagents for detecting and measuring in blood or in a fraction thereof the levels of any of the biomarkers mentioned above. More specifically, the biomarker is any of: CD38⁺ PBMCs, Tₕ2 cells, Tₕ2/Tₕ1 ratio, naïve CD4⁺ T cells, effector memory CD4⁺ T cells, naïve CD8⁺ T cells, effector memory CD8⁺ T cells level, and HLA-DR T cells, or combinations thereof. The kits may further include a container for holding a blood, plasma or serum sample, and printed instructions for detecting or measuring the biomarkers in the blood, plasma or serum sample. The reagents may be packaged in separate containers.

The reagents encompass any reagent capable of specifically identifying the target molecules (biomarkers), such as antibodies; antibody-derived products designed to specifically identify the specific molecules, such as, for example, a single-chain antibody fragment; and binding partners of the specific molecules. The term “antibody” relates to any form of antibodies such as polyclonal and monoclonal antibodies, as well as hybrid, chimeric, or humanized antibodies.

The reagents, e.g. antibodies, may be bound to a detectable moiety for detecting the binding of the reagent to the target molecule. Examples for detectable moieties include, but are not limited to fluorophores, chromophore, radio isotopes, ligands such as biotin, etc.

The kits may further include the appropriate agents for detecting the binding of the detectable moieties.

Accordingly, in a further aspect, the present invention provides a kit for early detection and diagnosis of a neurodegenerative disease, disorder, or condition in a subject at risk of developing or suspected of having the neurodegenerative disease, disorder, or condition, the kit comprising reagents for measuring in a blood sample at least one type of T cells selected from CD3⁺CD4⁺CD38⁺ (CD38 CD4⁺ T cells), CD3⁺CD8⁺ CD38⁺ (CD38 CD8⁺ T cells), CD3⁺CD4⁺CXCR5⁻CCR6⁻CXCR3⁻ (Tₕ2 cells), CD3⁺CD4⁺CXCR5⁻CCR6⁻CXCR3⁺ (Tₕ1 cells), CD3⁺CD4⁺ CXCR5⁻CCR6⁺CXCR3⁻ (Tₕ17 cells), CD3⁺CD4⁺CCR7⁺CD45RO⁻CD45RA⁺ (naïve CD4⁺ T cells), CD3⁺CD8⁺CCR7⁺CD45RO⁻CD45RA⁺ (naïve CD8⁺ T cells), CD3⁺CD4⁺ CCR7⁻CD45RO⁻CD45RA⁺ (Terminally differentiated effector memory T cells CD4⁺ T cells), CD45⁺ CD3⁺CD8⁺CCR7⁻CD45RO⁻CD45RA⁺ (Terminally differentiated effector memory T cells CD8⁺ T cells), CD3⁺CD4⁺CCR7⁺CD45RA⁻ (Central memory CD4⁺ T cells), CD3⁺CD8⁺ CCR7⁺CD45RA⁻ (Central memory CD8⁺ T cells), CD45⁺CD3⁺CD4⁺CCR7⁻CD45RA⁻ (Effector memory CD4⁺ T cells), CD45⁺CD3⁺CD8⁺ CCR7⁻CD45RA⁻ (Effector memory CD8⁺ T cells), CD45⁺ CD11c⁺ CD14⁺ CD16⁻ (classical monocytes), CD45⁺ CD19⁺ CD27⁺ (plasma cells), CD4⁺ HLA-DR⁺ T cells, and CD8⁺HLA-DR⁺ T cells.

In an additional aspect, the present invention provides a composition for early detection and diagnosis of a neurodegenerative disease, disorder, or condition in a subject at risk of developing or suspected of having the neurodegenerative disease, disorder, or condition, the composition comprising antibodies for measuring in a blood sample at least one type of T cells selected from CD3⁺CD4⁺ CD38⁺ (CD38 CD4⁺ T cells), CD3⁺CD8⁺ CD38⁺ (CD38 CD8⁺ T cells), CD3⁺CD4⁺ CXCR5⁻CCR6⁻CXCR3⁻ (Tₕ2 cells), CD3⁺CD4⁺ CXCR5⁻CCR6⁻CXCR3⁺ (Tₕ1 cells), CD3⁺CD4⁺ CXCR5⁻CCR6⁺CXCR3⁻ (Tₕ17 cells), CD3⁺CD4⁺CCR7⁺CD45RO⁻CD45RA⁺ (naïve CD4⁺ T cells), CD3⁺CD8⁺CCR7⁺CD45RO⁻CD45RA⁺ (naïve CD8⁺ T cells), CD3⁺CD4⁺ CCR7⁻CD45RO⁻CD45RA⁺ (Terminally differentiated effector memory T cells CD4⁺ T cells), CD45⁺CD3⁺CD8⁺CCR7⁻CD45RO⁻CD45RA⁺ (Terminally differentiated effector memory T cells CD8⁺ T cells), CD3⁺CD4⁺CCR7⁺CD45RA⁻ (Central memory CD4⁺ T cells), CD3⁺CD8⁺CCR7⁺CD45RA⁻ (Central memory CD8⁺ T cells), CD45⁺ CD3⁺CD4⁺CCR7⁻CD45RA⁻ (Effector memory CD4⁺ T cells), CD45⁺ CD3⁺CD8⁺ CCR7⁻CD45RA⁻ (Effector memory CD8⁺ T cells), CD45⁺ CD11c⁺ CD14+CD16⁻ (classical monocytes), CD45⁺ CD19⁺ CD27⁺ (plasma cells), CD4⁺HLA-DR⁺ T cells, and CD8⁺HLA-DR⁺ T cells.

In some embodiments, the kit or the composition comprises at least one antibody specific for CD3, at least one antibody specific for CD4, and at least one antibody specific for CD38, and at least one set of at least one antibody selected from the following sets: (i) at least one antibody specific for CXCR5, at least one antibody specific to CCR6, and at least one antibody specific to CXCR3; (ii) at least one antibody specific for CCR7, at least one antibody specific to CD45RO, and at least one antibody specific to CD45RA; (iii) at least one antibody specific for CD8, at least one antibody specific for CCR7, at least one antibody specific for CD45RO, and at least one antibody specific for CD45RA.

In some embodiments, the kit or the composition comprises at least one antibody specific for CD3, at least one antibody specific for CD4, and at least one antibody specific for CD38, for measuring the frequency of CD38⁺ cells out of total CD4⁺ cells.

In some embodiments, the kit or the composition comprises at least one antibody specific for CD3, at least one antibody specific for CD4, at least one antibody specific for CD38, and at least one antibody specific for CD8, for additionally measuring the frequency of CD38⁺ cells out of total CD8⁺ cells.

In some embodiments, the kit or the composition comprises at least one antibody specific for CD3, at least one antibody specific for CD4, at least one antibody specific for CXCR5, at least one antibody specific to CCR6, and at least one antibody specific to CXCR3, for measuring the Tₕ2/Tₕ1 ratio.

In some embodiments, the kit or the composition comprises at least one antibody specific for CD3, at least one antibody specific for CD4, at least one antibody specific for CCR7, at least one antibody specific to CD45RO, and at least one antibody specific to CD45RA, for measuring the frequency of naïve or activated CD4⁺ cells out of total CD4⁺ cells.

In some embodiments, the kit or the composition comprises at least one antibody specific for CD3, at least one antibody specific for CD4, and at least one antibody specific for IFNγ, for measuring the frequency of IFNγ-producing T cells.

In some embodiments, the kit or the composition comprises at least one antibody specific for CD3, at least one antibody specific for CD4, and at least one antibody specific for HLA-DR, for measuring the frequency of HLA-DR expressing T cells.

In some embodiments, the kit or the composition comprises at least one antibody specific for CD3, at least one antibody specific for CD4, at least one antibody specific for CD38, at least one antibody specific for CXCR5, at least one antibody specific to CCR6, and at least one antibody specific to CXCR3, for measuring CD38⁺ cells level out of total CD4⁺ cells and Tₕ2/Tₕ1 ratio.

In some embodiments, the kit or the composition comprises at least one antibody specific for CD3, at least one antibody specific for CD4, at least one antibody specific for CD38, at least one antibody specific for CCR7, at least one antibody specific to CD45RO, and at least one antibody specific to CD45RA, for measuring CD38⁺ cells level and the frequency of naïve or activated CD4⁺ cells out of total CD4⁺ cells.

In some embodiments, the kit or the composition comprises at least one antibody specific for CD3, at least one antibody specific for CD4, at least one antibody specific for CD38, at least one antibody specific for CD8, at least one antibody specific for CCR7, at least one antibody specific for CD45RO, and at least one antibody specific for CD45RA, for measuring CD38⁺ cells out of total CD4⁺ cells and further measuring the frequency of naïve or effector CD8⁺ cells out of total CD8⁺ cells.

In some embodiments, the kit or the composition comprises at least one antibody specific for CD3, at least one antibody specific for CD4, at least one antibody specific for CD38, and at least one antibody specific for HLA-DR, for measuring CD38⁺ cells out of total CD4⁺ and further out of HLA-DR⁺ T cells.

In some embodiments, the kit or the composition comprises at least one antibody specific for CD3, at least one antibody specific for CD4, at least one antibody specific for CD38, at least one antibody specific for CXCR5, at least one antibody specific to CCR6, and at least one antibody specific to CXCR3.

In some embodiments, the kit or the composition further comprises at least one antibody specific to CD45.

In some embodiments, the kit or the composition further comprises at least one antibody against at least one of CD66b, CD14, CD20, and TCRγδ, for negative identification of cells.

In some embodiments, the reagents or the antibodies are linked to a detectable moiety, such as a fluorophore.

In in a further aspect, the present invention provides a kit or a composition comprising at least one antibody specific for CD3, at least one antibody specific for CD4, and at least one antibody specific for CD38; and at least one antibody specific for a biomarker selected from the following biomarkers: CXCR5, CCR6, CXCR3, CCR7, CD45RO, CD45RA, CD8, CCR7, CD45RO, CD45RA, CD19, CD27, CD11c, CD14, CD16, IFNγ, GLUT1, HLA-DR, IL-10, IL-22, and IL-4.

Embodiment 1: a method for early detection or diagnosis of a neurodegenerative disease, disorder, or condition in a subject at risk of developing or suspected of having the neurodegenerative disease, disorder, or condition, the method comprising measuring in a blood sample obtained from the subject or a fraction thereof the levels of at least one biomarker selected from CD38⁺ peripheral blood mononuclear cells (PBMCs), trigonelline, GLUT1 expression in CD4⁺ T cells, Tₕ2, Tₕ2/Tₕ1 ratio, naïve T cells, adenosine, allose, and HLA-DR T cells, wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that the subject is likely developing, or affected by, said neurodegenerative disease, disorder, or condition.

Embodiment 2: The method of Embodiment 1, wherein the at least one biomarker comprises CD38⁺ PBMCs.

Embodiment 3: The method of Embodiment 2, wherein the increased level of CD38⁺ PBMCs compared to the respective reference for CD38⁺ PBMCs level is increased by at least about 10%, 15%, 20%, 25%, 30%, 40%, 50%, 60%, 70%, 80%, 90%, or 100%.

Embodiment 4: The method of Embodiment 2 or embodiment 3, wherein the CD38⁺ PBMCs are CD38⁺ CD4⁺ or CD38⁺ CD8⁺ T cells, and their level is calculated by measuring the levels of (1) CD38⁺ CD4⁺ cells and (2) total CD4⁺ cells, and calculating the proportion of (1) out of (2), or measuring the levels of (1) CD38⁺CD8⁺ cells and (2) total CD8⁺ cells, and calculating the proportion of (1) out of (2), respectively.

Embodiment 5: The method of Embodiment 4, wherein the at least one biomarker comprises CD38⁺ CD4⁺ T cells and CD38⁺ CD8⁺ T cells.

Embodiment 6: The method of Embodiment 4 or 5, wherein the respective reference for CD38⁺ CD4⁺ T cells out of total CD4⁺ T cells is between about 35% and about 40%, and the respective reference for CD38⁺ CD8⁺ T cells out of total CD8⁺ T cells is between about 15% and about 25%.

Embodiment 7: The method of any one of Embodiments 1 to 6, wherein the at least one biomarker comprises Tₕ2 cells and/or Tₕ2/Tₕ1 ratio.

Embodiment 8: The method of Embodiment 7, wherein the level of the Tₕ2 cells is measured by measuring the level of CXCR5⁻CCR6⁻CXCR3⁻CD4⁺ cells, and/or the Tₕ2/Tₕ1 ratio is calculated by measuring the level of (1) CXCR5⁻CCR6⁻CXCR3⁻CD4⁺ cells (Tₕ2), and (2) CXCR5⁻CCR6⁻CXCR3⁺CD4⁺ cells (Tₕ1), and calculating the proportion of (1) out of (2).

Embodiment 9: The method of Embodiment 7 or 8, wherein the increased Tₕ2/Tₕ1 ratio is increased by at least about 30%, 35%, 40%, 45%, 50%, 60%, or 70% compared to the respective reference for Tₕ2/Tₕ1 ratio.

Embodiment 10: The method of any one of Embodiments 7 to 9, wherein the respective reference for Tₕ2/Tₕ1 ratio is at least about 10, 11, 12, 13, or 14.

Embodiment 11: The method of any one of Embodiments 1 to 10, wherein the at least one biomarker comprises naïve CD4⁺ T cells level.

Embodiment 12: The method of Embodiment 11, wherein the naïve CD4⁺ T cells level is calculated by measuring the level of (1) CCR7⁺CD45RO⁻CD45RA⁺ (naïve) CD4⁺ T cells, and (2) total CD4⁺ T cells, and calculating the proportion of (1) out of (2).

Embodiment 13: The method of Embodiment 11 or 12, wherein the increased level of naïve CD4⁺ T cells compared to the respective reference is increased by at least about 30%, 35%, 40%, 45, or 50%.

Embodiment 14: The method of any one of Embodiments 1 to 13, wherein the at least one biomarker comprises GLUT1 expression in CD4⁺ T cells.

Embodiment 15: The method of any one of Embodiments 1 to 14, wherein the at least one biomarker comprises plasma levels of at least one metabolite selected from trigonelline, allose, and adenosine.

Embodiment 16: The method of Embodiment 15, wherein the biomarker comprises CD38⁺ PBMCs and at least one metabolite selected from trigonelline, allose, and adenosine.

Embodiment 17: The method of Embodiment 16, wherein the at least one biomarker comprises CD38+ PBMCs and trigonelline.

Embodiment 18: The method of Embodiment 14, wherein the at least one biomarker comprises CD38+ PBMCs and GLUT1 expression in CD4⁺ T cells.

Embodiment 19: The method of Embodiment 7, wherein the at least one biomarker comprises CD38⁺ PBMCs and Tₕ2/Tₕ1 ratio.

Embodiment 20: The method of Embodiment 17, wherein increased levels of CD38+ PBMCs compared to a respective reference and decreased levels of trigonelline as compared to a respective reference indicate that the subject is likely developing, or affected by, said neurodegenerative disease, disorder, or condition.

Embodiment 21: The method of Embodiment 18, wherein increased levels of CD38+ PBMCs compared to a respective reference and decreased levels of GLUT1 expression in CD4⁺ T cells as compared to a respective reference indicate that the subject is likely developing, or affected by, said neurodegenerative disease, disorder, or condition.

Embodiment 22: The method of any one of Embodiments 1 to 21, wherein the level of plasma neurofilament light chain (NfL) in the subject is not increased above about 20 pg/ml.

Embodiment 23: The method of any one of Embodiments 1 to 22, wherein said neurodegenerative disease, disorder, or condition is selected from Alzheimer's disease (AD), amyotrophic lateral sclerosis (ALS), Parkinson's disease, Huntington's disease, primary progressive multiple sclerosis; secondary progressive multiple sclerosis, attention deficit disorder (ADD), corticobasal degeneration, Creutzfeldt-Jakob disease (CJD), Rett syndrome, a retinal degeneration disorder selected from the group consisting of age-related macular degeneration and retinitis pigmentosa; anterior ischemic optic neuropathy; glaucoma; uveitis; depression; trauma-associated stress or post-traumatic stress disorder, frontotemporal dementia (FTD), Lewy body dementias, mild cognitive impairments, posterior cortical atrophy, primary progressive aphasia or progressive supranuclear palsy.

Embodiment 24: The method of Embodiment 23, wherein said neurodegenerative disease, disorder, or condition is AD.

Embodiment 25: A method for early detection or diagnosis of Alzheimer's disease (AD) in a subject at risk of developing or suspected of having AD, the method comprising measuring in a blood sample obtained from the subject or a fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells, wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicate that the subject is likely developing, or affected by, AD.

Embodiment 26: A method for stratifying a subject in a subgroup of a clinical trial of an immune checkpoint modulator for the treatment of a neurodegenerative disease, disorder, or condition, the method comprising measuring in a blood sample obtained from the subject or a fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells, wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that that the subject is likely to be responsive to treatment with the immune checkpoint modulator, and no change or a decreased level of at least one of CD38⁺ PBMCs, Tₕ2, Tₕ2/Tₕ1 ratio, naïve T cells, and adenosine, as compared to a respective reference, and/or no change or an increased level of at least one of trigonelline, GLUT1 expression in CD4⁺ T cells, allose, and HLA-DR T cells as compared to a respective reference, indicates that that the subject is likely not to be responsive to treatment with the immune checkpoint modulator, and said biomarker enables the stratification of said subject into a subgroup for said clinical trial.

Embodiment 27: A method of assessing efficacy of a treatment by an immune checkpoint modulator in a subject diagnosed with a neurodegenerative disease, disorder, or condition, said method comprising:
obtaining a first blood sample from the subject;
administering the immune checkpoint modulator to the subject;
obtaining a second blood sample from the subject;
measuring in the first blood sample or a fraction thereof and in the second blood sample or fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells, and comparing the level of the at least one biomarker in the first blood sample with the level of the biomarker in the second blood sample; wherein a decreased level of at least one of CD38⁺ PBMCs, Tₕ2, Tₕ2/Tₕ1 ratio, naïve T cells, and adenosine, and/or an increased level of at least one of trigonelline, GLUT1 expression in CD4⁺ T cells, allose, and HLA-DR T cells in the second blood sample compared to the first blood sample indicates that the treatment by an immune checkpoint modulator is likely effective.

Embodiment 28: A method for treating or preventing a neurodegenerative disease, disorder, or condition in a subject, said method comprising measuring in a blood sample or fraction thereof the levels of at least one biomarker selected from CD38⁺ PBMCs, trigonelline, GLUT1 expression in CD4⁺ T cells, Tₕ2, Tₕ2/Tₕ1 ratio, naïve T cells, adenosine, allose, and HLA-DR T cells, wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that the subjects is likely developing, or is affected by, the neurodegenerative disease, disorder, or condition, and the method further comprising administering an immune checkpoint modulator to the subject which is likely developing, or is affected by, the neurodegenerative disease, disorder, or condition, thereby treating the subject.

Embodiment 29: A kit comprising at least one antibody specific for CD3, at least one antibody specific for CD4, and at least one antibody specific for CD38; and at least one antibody specific for a biomarker selected from the following biomarkers: CXCR5, CCR6, CXCR3, CCR7, CD45RO, CD45RA, CD8, CCR7, CD45RO, CD45RA, CD19, CD27, CD11c, CD14, CD16, IFNγ, GLUT1, HLA-DR, IL-10, IL-22, and IL-4.

For purposes of clarity, and in no way limiting the scope of the teachings, unless otherwise indicated, all numbers expressing quantities, percentages or proportions, and other numerical values recited herein, should be interpreted as being preceded in all instances by the term “about”, regardless of whether “about” is explicitly prepended to the numerical value. Accordingly, the numerical parameters recited in the present specification are approximations that may vary depending on the desired outcome. For example, each numerical parameter may be construed in light of the number of reported significant digits and by applying ordinary rounding techniques. Accordingly, unless indicated to the contrary, the numerical parameters set forth in this specification are approximations that may vary by up to plus or minus 10% depending upon the desired properties to be obtained by the present invention.

Additionally for purposes of clarity, it is noted that where measuring the “level” of a biomarker requires to calculate its level out of (divide by) the level of something else, such as a different cell population, or another biomarker, then the term “level” is intended to mean “relative level”.

Unless otherwise indicated, all numbers used in this specification are to be understood as being modified in all instances by the term “about”. Accordingly, unless indicated to the contrary, the numerical parameters set forth in this specification are approximations that may vary by up to 10% higher or lower depending upon the desired properties to be obtained by the present invention.

For any embodiment above, the term “comprises” or “comprising” may be replaced by the term “consisting of”.

The invention will now be illustrated by the following non-limiting examples.

### EXAMPLES

### Material and Methods

### Human Samples

All samples from human subjects were obtained via the Galilee Medical Center and experimental procedures carried out in accordance with the guidelines of the Weizmann Institute of Science Institutional Review Board. Written informed consent was obtained from all subjects, and the Institutional Review Board of the Galilee Medical Center approved the study. Fresh whole human peripheral blood samples were collected in EDTA collection tubes from a cohort of individuals, including females and males aged 29-68 years, who carry an autosomal dominant APP duplication and already developed clinically diagnosed symptomatic familial Alzheimer's disease. Samples were also collected from pre-symptomatic patients, who carry the same genetic rearrangement, but do not fulfill yet the diagnostic criteria, and from individuals from the same family in which genetic testing did not show the APP duplication.

### Plasma Samples

Blood samples were centrifuged at 2000 G at 4° C. for 10 min within approximately 5 h of collection. Plasma supernatant was collected, divided into aliquots, and frozen at −80° C. until further NfL, untargeted or targeted metabolic determination.

### Untargeted Metabolomics

To remove protein, dissociated small molecules bound to protein or trapped in the precipitated protein matrix, and to recover chemically diverse metabolites, proteins were precipitated with methanol and the resulted extract was analyzed by mass spectrometry. Compounds were identified by comparison to library entries of purified standards or recurrent unknown entities.

### Targeted Metabolomics

To remove protein, dissociated small molecules bound to protein or trapped in the precipitated protein matrix, and to recover chemically diverse metabolites, proteins were precipitated with methanol and the resulted extract was analyzed by mass spectrometry. Compounds were identified by comparison to library entries of purified standards or recurrent unknown entities.

### Mass Cytometry

Panels have been designed and optimized for deep immune profiling of human peripheral blood mononuclear cells, through comprehensive identification and characterization of key immune cell populations, including all major T cell subsets (CD4⁺ and CD8⁺ naïve, central memory, effector and effector memory), CD4⁺ regulatory T cells, B cell subsets (naïve, memory, and transitional), plasmablasts, natural killer cells, T helper (T_{H}) cell subsets (T_{H}1, T_{H}2, T_{H}17, and T_{H}22), γδ T cells, MAIT T cells, monocytes, dendritic cells (plasmacytoid and myeloid), and neutrophils. Briefly, all individual samples were incubated with Human TruStain FcX™ (BioLegend).

For the Human Immune Monitoring panel (HIM), individual samples stained with a panel of surface antibodies for 30 min at RT and washed with Maxpar® Cell Staining Buffer. Individual samples were then fixated with Fix I Buffer, and permeabilized with Barcode Perm Buffer. Cell-ID™ cisplatin viability stain was used prior to cell barcoding of samples with Cell-ID™ Palladium Barcoding Kit for 45 min at RT, after which the samples were washed with Maxpar® Barcode Perm Buffer and finally combined into a composite sample.

For the Cytokines/Transcription Factors (TFs) panel, individual samples were stimulated or not with PMA (100 ng/ml) plus ionomycin (1 μg/ml), in the presence of Brefeldin A and Monensin for 5 h at 37° C. Cell-ID™ cisplatin viability stain was used prior to staining of the samples with a panel of surface antibodies for 30 min at RT and a wash with Maxpar® Cell Staining Buffer. Samples were then fixated with Foxp3 Fixation/Permeabilization solution (eBioscience™) and permeabilized with Foxp3 Permeabilization buffer (eBioscience™), prior to cell barcoding with Cell-ID™ Palladium Barcoding Kit for 45 min at RT, after which the samples were washed with Foxp3 Permeabilization buffer and finally combined into a composite sample. This sample was then incubated with a panel of intracellular antibodies for 60 min at RT and washed with Foxp3 Permeabilization buffer.

For the Immunometabolic panel, we used Cell-ID™ cisplatin viability stain prior to live-cell barcoding of individual samples with CD45-cadmium labeled antibodies and combination into a composite sample. The composite sample was then incubated with a panel of surface antibodies for 30 min at RT, washed with Maxpar® Cell Staining Buffer and fixated with 1.6% formaldehyde. Following permeabilization with ice-cold methanol, this sample was then incubated with a panel of intracellular antibodies for 60 min at RT and washed with Maxpar® Cell Staining Buffer.

After washing, composite samples were incubated with formaldehyde 4% overnight at 4° C. Prior to acquisition in a Helios™ II CyTOF® system, samples were incubated with Cell-ID™ Intercalator-Ir for 20 min, washed with Maxpar® Cell Staining Buffer, Maxpar® PBS and Maxpar® mass cytometry grade water.

### Data Workflow

After acquisition, data from acquired samples was bead-normalized using Fluidigm's software, and barcoded cells were assigned back to their initial samples upon debarcoding. Normalized data was then uploaded onto the Cytobank analysis platform to perform initial CD45⁺ live immune cells gating. For further downstream analysis, pre-gated data was imported into FlowJo™ software. Figures were prepared in Photoshop (Adobe).

TABLE 1
CyTOF (mass spec) antibodies

| Target | Supplier | Clone | Metal | Location | Panel |
|---|---|---|---|---|---|
| αCD45 | Fluidigm | HI30 | 89Y | Surface | HIM |
| αCD45 | Fluidigm | HI30 | 89Y | Surface | Cytokines/TFs |
| αCD196/CCR6 | Fluidigm | G034E3 | 141Pr | Surface | HIM |
| αSIRT3 | Abcam | EPR19755 | 141Pr | Intracellular | Immunometabolic |
| αCD19 | Fluidigm | HIB19 | 142Nd | Surface | HIM |
| αCD19 | Fluidigm | HIB19 | 142Nd | Surface | Cytokines/TFs |
| αCD19 | Fluidigm | HIB19 | 142Nd | Surface | Immunometabolic |
| αCD127/IL7-R | Fluidigm | A019D5 | 143Nd | Surface | HIM |
| αCD127/IL7-R | Fluidigm | A019D5 | 143Nd | Surface | Cytokines/TFs |
| αGLUT1 | Abcam | EPR3915 | 143Nd | Intracellular | Immunometabolic |
| αCD38 | Fluidigm | HIT2 | 144Nd | Surface | HIM |
| αCD38 | Fluidigm | HIT2 | 144Nd | Intracellular | Immunometabolic |
| αIL-4 | Fluidigm | MP4-25D2 | 144Nd | Intracellular | Cytokines/TFs |
| αXBP1 | Invitrogen | PA5-27650 | 145Nd | Intracellular | Immunometabolic |
| αlgD | Fluidigm | IA6-2 | 146Nd | Surface | HIM |
| αTNFα | Fluidigm | MAb11 | 146Nd | Intracellular | Cytokines/TFs |
| αCD8 | Fluidigm | RPA-T8 | 146Nd | Surface | Immunometabolic |
| αCD11c | Fluidigm | Bu15 | 147Sm | Surface | HIM |
| αCD11c | Fluidigm | Bu15 | 147Sm | Surface | Cytokines/TFs |
| αGAPDH | Invitrogen | 6C5 | 147Sm | Intracellular | Immunometabolic |
| αCD16 | Fluidigm | 3G8 | 148Nd | Surface | HIM |
| αIL-17A | Fluidigm | BL168 | 148Nd | Intracellular | Cytokines/TFs |
| αS6_p | BioLegend | A17020B | 148Nd | Intracellular | Immunometabolic |
| αCD194/CCR4 | Fluidigm | L291H4 | 149Sm | Surface | HIM |
| αCD45RO | Fluidigm | UCHL1 | 149Sm | Surface | Cytokines/TFs |
| αCD25 | Fluidigm | 2A3 | 149Sm | Surface | Immunometabolic |
| αIL-22 | Fluidigm | 22URTI | 150Nd | Intracellular | Cytokines/TFs |
| αTFAM | BioLegend | 18G102B2E11 | 150Nd | Intracellular | Immunometabolic |
| αCD123/IL-3R | Fluidigm | 6H6 | 151Eu | Surface | HIM |
| αIL-5 | Fluidigm | TRFK5 | 151Eu | Intracellular | Cytokines/TFs |
| αSARM1 | BioLegend | W16079A | 151Eu | Intracellular | Immunometabolic |
| αTCRγδ | Fluidigm | 11F2 | 152Sm | Surface | HIM |
| αTCRγδ | Fluidigm | 11F2 | 152Sm | Surface | Cytokines/TFs |
| αTCRγδ | Fluidigm | 11F2 | 152Sm | Surface | Immunometabolic |
| αCD185/CXCR5 | Fluidigm | RF8B2 | 153Eu | Surface | HIM |
| αTCR Vα7.2 | Fluidigm | 3C10 | 153Eu | Surface | HIM |
| αMCT1 | Invitrogen | P14612 | 153Eu | Surface | Immunometabolic |
| αCD3 | Fluidigm | UCHT1 | 154Sm | Surface | HIM |
| αCD3 | Fluidigm | UCHT1 | 154Sm | Surface | Cytokines/TFs |
| αCD3 | Fluidigm | UCHT1 | 154Sm | Surface | Immunometabolic |
| αCD45RA | Fluidigm | HI100 | 155Gd | Surface | HIM |
| αCD45RA | Fluidigm | HI100 | 155Gd | Surface | Cytokines/TFs |
| αHIF-1α | Invitrogen | 16H4L13 | 155Gd | Intracellular | Immunometabolic |
| αIL-6 | Fluidigm | MQ2-13A5 | 156Gd | Intracellular | Cytokines/TFs |
| αNLRP3 | R&D | 652519 | 156Gd | Intracellular | Immunometabolic |
| αPARP1 | Abcam | EPR18461 | 157Gd | Intracellular | Immunometabolic |
| αCD27 | Fluidigm | L128 | 158Gd | Surface | HIM |
| αIL-2 | Fluidigm | MQ1-17H12 | 158Gd | Intracellular | Cytokines/TFs |
| αATP5A | Abcam | 15H4C4 | 158Gd | Intracellular | Immunometabolic |
| αCD197/CCR7 | Fluidigm | G043H7 | 159Tb | Surface | Cytokines/TFs |
| αCD197/CCR7 | Fluidigm | G043H7 | 159Tb | Surface | Immunometabolic |
| αCD28 | Fluidigm | CD28.2 | 160Gd | Surface | HIM |
| αCD14 | Fluidigm | RMO52 | 160Gd | Surface | Cytokines/TFs |
| αCD39 | Fluidigm | A1 | 160Gd | Surface | Immunometabolic |
| αTbet | Fluidigm | 4B10 | 161Dy | Intracellular | Cytokines/TFs |
| αTbet | Fluidigm | 4B10 | 161Dy | Intracellular | Immunometabolic |
| αCD66b | Fluidigm | 80H3 | 162Dy | Surface | HIM |
| αFoxp3 | Fluidigm | 259D/C7 | 162Dy | Intracellular | Cytokines/TFs |
| αVDAC | Abcam | 20B12AF2 | 162Dy | Intracellular | Immunometabolic |
| αCD183/CXCR3 | Fluidigm | G025H7 | 163Dy | Surface | HIM |
| αTGFβ | Fluidigm | TW4-6H10 | 163Dy | Intracellular | Cytokines/TFs |
| αCD56 | Fluidigm | NCAM-16.2 | 163Dy | Surface | Immunometabolic |
| αCD161 | Fluidigm | HP-3G10 | 164Dy | Surface | HIM |
| αArginase | Fluidigm | 14D2C43 | 164Dy | Intracellular | Cytokines/TFs |
| αCD45RO | Fluidigm | UCHL1 | 164Dy | Surface | Immunometabolic |
| αCD45RO | Fluidigm | UCHL1 | 165Ho | Surface | HIM |
| αIFNγ | Fluidigm | B27 | 165Ho | Intracellular | Cytokines/TFs |
| αPGC1α | R&D | S571 | 165Ho | Intracellular | Immunometabolic |
| αCD24 | Fluidigm | ML5 | 166Er | Surface | HIM |
| αIL-10 | Fluidigm | JES3-9D7 | 166Er | Intracellular | Cytokines/TFs |
| αGLUT1 | Abcam | EPR3915 | 166Er | Surface | Immunometabolic |
| αCD197/CCR7 | Fluidigm | G043H7 | 167Er | Surface | HIM |
| αGATA3 | Fluidigm | TWAJ | 167Er | Intracellular | Cytokines/TFs |
| αGATA3 | Fluidigm | TWAJ | 167Er | Intracellular | Immunometabolic |
| αCD8 | Fluidigm | SK1 | 168Er | Surface | Cytokines/TFs |
| αCD127/IL-7R | Fluidigm | A019D5 | 168Er | Surface | Immunometabolic |
| αCD25 | Fluidigm | 2A3 | 169Tm | Surface | Cytokines/TFs |
| αLDH | Abcam | EP1566Y | 169Tm | Intracellular | Immunometabolic |
| αRORγ | Fluidigm | 600214 | 170Er | Intracellular | Cytokines/TFs |
| αCD45RA | Fluidigm | HI100 | 170Er | Surface | Immunometabolic |
| αCD20 | Fluidigm | 2H7 | 171Yb | Surface | HIM |
| αGranzyme B | Fluidigm | GB11 | 171Yb | Intracellular | Cytokines/TFs |
| αHK | Abcam | EPR20839 | 171Yb | Intracellular | Immunometabolic |
| αCD38 | Fluidigm | HIT2 | 172Yb | Surface | Cytokines/TFs |
| αCD38 | Fluidigm | HIT2 | 172Yb | Surface | Immunometabolic |
| αHLA-DR | Fluidigm | L243 | 173Yb | Surface | HIM |
| αHLA-DR | Fluidigm | L243 | 173Yb | Surface | Cytokines/TFs |
| αCD4 | Fluidigm | SK3 | 174Yb | Surface | HIM |
| αCD4 | Fluidigm | SK3 | 174Yb | Intracellular | Cytokines/TFs |
| αCD4 | Fluidigm | SK3 | 174Yb | Surface | Immunometabolic |
| αPerforin | Fluidigm | B-D48 | 175Yb | Intracellular | Cytokines/TFs |
| αCD14 | Fluidigm | M5E2 | 175Lu | Surface | HIM |
| αPD-1 | Fluidigm | EH12.2H7 | 175Lu | Surface | Immunometabolic |
| αCD56 | Fluidigm | NCAM16.2 | 176Yb | Surface | HIM |
| αCD56 | Fluidigm | N901 | 176Yb | Surface | Cytokines/TFs |
| αc-Myc | Fluidigm | 9E10 | 176Yb | Intracellular | Immunometabolic |
| αCD11b | Fluidigm | ICRF44 | 209Bi | Surface | Cytokines/TFs |
| αCD11b | Fluidigm | ICRF44 | 209Bi | Surface | Immunometabolic |

### Example 1: Familial Alzheimer's Disease Carriers Present an Immune Deviation that Precedes Symptoms Onset

Blood samples were centrifuged, and plasma supernatant collected for further untargeted metabolic assessment. Peripheral blood mononuclear cells were isolated after Ficoll density gradient centrifugation, stained with a panel of surface and intracellular antibodies (see Table 1), and subsequently assessed by single-cell mass cytometry (CyTOF). Plasma neurofilament light chain (NfL) concentration was measured using Simoa® platform (Quanterix). Samples were randomized, blinded and measured in triplicates.

The inventors found that the Tₕ2/Tₕ1 profile ratio (CXCR3⁻CCR6⁻ representing Tₕ2, and CXCR3⁺CCR6⁻ representing Tₕ1, out of total Tₕ cells (CD45⁺CD3⁺CD4⁺CXCR5⁻)) to be increased in AD pre-symptomatic APP duplication carriers compared to control individuals, indicating that changes appear before appearance of AD symptoms (based on FIG. 1B). An increase in plasma neurofilament light chain (NfL) concentration (indicative of neuronal loss) was only seen in symptomatic AD patients, and not in pre-symptomatic carriers, (FIG. 1F). It was found that the increase in NfL was positively correlated with plasma top significant metabolites, creatine, N-acetylneuraminate, and 2-propylpentanoic acid (data not shown). FIG. 1D shows an increase in naïve (CCR7⁺CD45RO⁻CD45RA⁺) CD4⁺ cells out of the total CD4⁺ cells in AD carriers compared to healthy individuals, and FIG. 1E shows a decrease in effector memory (T_{EM}, CCR7⁻CD45RA⁻) CD8⁺ cells out of the total CD8⁺ cells in AD carriers, compared to healthy individual. FIG. 1A shows an overall reduction in CD4⁺ and CD8⁺ cells in the AD pre-symptomatic carriers.

### Example 2: Familial Alzheimer's Disease Carrier Present an Increase in Metabolically Dysfunctional CD38-Expressing T Cells

Blood samples were centrifuged, and plasma supernatant collected for further untargeted metabolic assessment. Peripheral blood mononuclear cells were isolated after Ficoll density gradient centrifugation, stained with a panel of surface antibodies (see Table 1), and subsequently assessed by single-cell mass cytometry (CyTOF).

As shown in FIGS. 2A and 2B, the frequency of CD38^{hi} T cells out of CD4⁺ and CD8⁺ T cell populations is increased in AD carriers compared to controls.

### Example 3: Pre-Symptomatic AD Individuals Feature a Selective Metabolic Impairment in CD4⁺ T Cells

Principal Component Analysis (PCA) (presented in FIGS. 3A and 3B) is a multivariate technique that is used to reduce the dimension of a dataset while retaining as much information from the data as possible. The “information” in the data is given by the variance. The first principal component (PC1) “explains” (or accounts for) the majority of the variance in the data. The second PC (PC2) “explains” the next most amount of variance in the data.

From FIGS. 4C and 3D it can be seen that surface expression of GLUTs was reduced in CD4⁺ T cells and not in CD8⁺ T cells, as determined by mass cytometry.

Additionally, as can be seen from FIGS. 4B, 4C, and 4E, the levels of allose (a major metabolite of fructose/mannose), and of trigonelline (a major metabolite of nicotinate and nicotinamide) are decreased in plasma from AD carriers compared to controls. As seen in FIG. 4G, the level of plasma adenosine is increased in plasma from AD carriers. In addition, the following metabolites were somewhat decreased in plasma from pre-symptomatic individuals compared to controls, and decreased further in symptomatic patients: citrate, 2, 6-dihydroxybenzoic acid; hypotaurine; and indoleacetaldehyde (data not shown). Finally, the levels of choline glycerophosphate were decreased in symptomatic patients, and the levels of creatine, N-acetylneuraminate, and 2-propylpentanoic acid were increased in symptomatic patients (data now shown).

### Example 4: Artificial Intelligence Analysis of Biomarker Combinations

Table 2 presents the predictive performance results by a Gaussian Naïve Bayes analysis on three biomarker combinations, namely: 1) CD4⁻CD38 and CD8⁻CD38 (representing CD38⁺ CD4 and CD8 T cells); 2) CD4−CD38+CD8−CD38+CD4−CD45RA+CD8−CD45RA (representing CD38⁺ CD4 and CD8 T cells with naïve CD4 and CD8 cells); and 3) CD4−CD38+CD8−CD38+“Classical monocytes CD38”+“Plasma cells CD38” (representing CD38⁺ CD4 cells, CD8 cells, classical monocytes, and plasma cells), using the same AD/healthy subjects population of the previous examples.

Naïve Bayes tends to outperform other learning algorithms regardless of the feature selection algorithm. Despite its simplicity, Naïve Bayes has important advantages: it is not prone to over-fitting, and it reaches its asymptotic accuracy with a much smaller training set, compared to other ML methods. The 95% confidence intervals were calculated based on the 100 repetitions (when applicable) or by using their approximation. The sensitivity and specificity were estimated using a default cut-off of 0.5.

As can be seem from Table 2, all three biomarkers have rather high sensitivity (above −0.7 or 0.8). Specificity of the third group was also above 0.8. It is noted that the sensitivity and specificity value are not so high due to the small number of individuals in the samples and are expected to increase when a larger sample size is used.

TABLE 2
AI analysis of biomarker combinations by Gaussian Naive Bayes

| biomarkers | AUC | Sensitivity | Specificity |
|---|---|---|---|
| CD4 − CD38 + CD8 − CD38 | 0.743 ± | 0.636 ± | 0.643 ± |
| | 0.17 | 0.16 | 0.16 |
| CD4 − CD38 + CD8 − CD38 + CD4 − | 0.713 ± | 0.576 ± | 0.647 ± |
| CD45RA + CD8 − CD45RA | 0.18 | 0.17 | 0.16 |
| CD4 − CD38 + CD8 − CD38 + | 0.835 ± | 0.813 ± | 0.765 ± |
| “Classical monocytes CD38” + | 0.14 | 0.13 | 0.14 |
| “Plasma cells CD38” | | | |

TABLE 3
Gaussian parameters of the best combination

| | | Patients | | |
|---|---|---|---|---|
| Biomarker | Parameter | AD | Healthy | p-value |
| CD4 − CD38 | mean | 11.5675 | 7.1932 | 0.011 |
| | std. dev. | 4.4648 | 4.4537 | |
| CD8 − CD38 | mean | 5.7639 | 3.0801 | 0.004 |
| | std. dev. | 2.6315 | 1.7568 | |
| Plasma − CD38 | mean | 1160.3496 | 911.0515 | 0.007 |
| | std. dev. | 254.5931 | 214.8075 | |
| Classical monocytes − | mean | 102.5156 | 82.4294 | 0.025 |
| CD38 | std. dev. | 26.1185 | 11.864 | |

The parameters μₖ and σₖ² for each variable and class. The p-values were calculated using the Kruskal-Wallis test. All four variables are found to be statistically significant.

As can be seen from Table 3, all biomarkers which in combination provided the best sensitivity and specificity values individually show an increase in the AD pre-symptomatic population compared to healthy individuals.

### REFERENCES

Arbore G, West E E, Spolski R, Robertson A A B, Klos A, Rheinheimer C, et al. T helper 1 immunity requires complement-driven NLRP3 inflammasome activity in CD4⁺ T cells. Science. 2016 Jun. 17; 352(6292):aad1210.
Baruch K, Ron-Harel N, Gal H, Deczkowska A, Shifrut E, Ndifon W. Mirlas-Neisberg N, Cardon M, Vaknin I, Cahalon L. Berkutzki T, Mattson M P, Gomez-Pinilla F, Friedman N, Schwartz M (2013) CNS-specific immunity at the choroid plexus shifts toward destructive Tₕ2 inflammation in brain aging. Proceedings of the National Academy of Sciences of the United States of America 110: 2264-2269
Baruch, K., Kertser, A., Porat, Z., and Schwartz, M. (2015a). Cerebral nitric oxide represses choroid plexus NFkB-dependent gateway activity for leukocyte trafficking. EMBO J. 34, 1816-1828.
Baruch, K, Rosenzweig, N., Kertser, A., Deczkowska, A., Sharif, A. M., Spinrad, A., Tsitsou-Kampeli, A., Sarel, A., Cahalon, L., and Schwartz, M. (2015b). Breaking immune tolerance by targeting Foxp3(⁺) regulatory T cells mitigates Alzheimer's disease pathology. Nat Commun 6, 7967.
Baruch and Schwartz (2013) CNS-specific T cells shape brain function via the choroid plexus. Brain Behav Immun. 2013 November; 34:11-6
Baruch K, Deczkowska A. David E. Castellano J M. Miller O. Kertser A, Berkutzki T, Bamett-Itzhaki Z, Bezalel D, Wyss-Coray T, Amit I, Schwartz M (2014) Aging. Aging-induced type I interferon response at the choroid plexus negatively affects brain function. Science 346: 89-93.
Baruch, K., Deczkowska, A., Rosenzweig, N., Tsitsou-Kampeli, A., Sharif, A. M., Matcovitch-Natan, O., Kertser, A., David, E., Amit, I., and Schwartz, M. (2016). PD-1 immune checkpoint blockade reduces pathology and improves memory in mouse models of Alzheimer's disease. Nat. Med. 22, 135-137.
Bowers E M, Yan G. Mukherjee C, Orry A, Wang L, Holbert M A, Crump N T. Hazzalin C A, Liszczak G, Yuan H, Larocca C, Saldanha S A, Abagyan R, Sun Y, Meyers D J, Marmorstein R. Mahadevan L C, Alani R M, Cole P A (2010) Virtual ligand screening of the p300/CBP histone acetyltransferase: identification of a selective small molecule inhibitor. Chemistry & biology 17: 471-482
Brestoff J R, Artis D (2013) Commensal bacteria at the interface of host metabolism and the immune system. Nature immunology 14: 676-684.
Buck M D, O'Sullivan D, Klein Geltink R I, Curtis J D, Chang C-H, Sanin D E, et al. Mitochondrial Dynamics Controls T Cell Fate through Metabolic Programming. Cell. 2016 Jun. 30; 166(1):63-76.
Camacho-Pereira et al., 2016, “CD38 dictates age-related NAD decline and mitochondrial dysfunction through a SIRT3-dependent mechanism”, Cell. Metab. 23(6): 1127-1139.
Chini C C S, Peclat T R, Wamer G M, Kashyap S, Espindola-Netto J M, de Oliveira G C, et al. CD38 ecto-enzyme in immune cells is induced during aging and regulates NAD⁺ and NMN levels. Nat Metab. 2020 Nov. 16:2(11):1284-304.
Clifford R. Jack Jr. et al. “Introduction to the recommendations from the National Institute on Aging—Alzheimer's Association workgroups on diagnostic guidelines for Alzheimer's disease.” Alzheimer's & Dementia: The Journal of the Alzheimer's Association 2011; 7(3):257-262;
Dalotto-Moreno T, Croci D O, Cerliani J P, Martinez-Allo V C, Dergan-Dylon S, Mendez-Huergo S P, Stupirski J C, Mazal D. Osinaga E, Toscano M A, Sundblad V, Rabinovich G A, Salatino M (2013) Targeting galectin-1 overcomes breast cancer-associated immunosuppression and prevents metastatic disease. Cancer research 73: 1107-1117
Ghiringhelli F, Bruchard M, Chalmin F, Rebe C (2012) Production of adenosine by ectonucleotidase: a key factor in tumor immunoescape. Journal of biomedicine & biotechnology 2012: 473712
Giau, V. V., Bagyinszky, E., Yang, Y. et al. Genetic analyses of early-onset Alzheimer's disease using next generation sequencing. Sci Rep 9, 8368 (2019).
Guy M. McKhann et al. “The diagnosis of dementia due to Alzheimer's disease: Recommendations from the National Institute on Aging—Alzheimer's Association workgroups on diagnostic guidelines for Alzheimer's disease.” Alzheimer's & Dementia: The Journal of the Alzheimer's Association 2011; 7(3):263-269; Marilyn S. Albert et al. “The diagnosis of mild cognitive impairment due to Alzheimer's disease: Recommendations from the National Institute on Aging—Alzheimer's Association workgroups on diagnostic guidelines for Alzheimer's disease.” Alzheimer's & Dementia: The Journal of the Alzheimer's Association 2011; 7(3):270-279;
Hartmann F J, Babdor J, Gherardini P F, Amir E-A D, Jones K, Sahaf B, et al. Comprehensive immune monitoring of clinical trials to advance human immunotherapy. Cell Rep. 2019 Jul. 16:28(3):819-831.e4.
Hirayama M. Nishikawa H, Nagata Y. Tsuji T, Kato T, Kageyama S, Ueda S, Sugiyama D, Hori S, Sakaguchi S. Ritter G, Old L J, Gnjatic S, Shiku H (2013) Overcoming regulatory T-cell suppression by a lyophilized preparation of Streptococcus pyogenes. European journal of immunology 43: 989-1000
Kunis G, Baruch K, Miller O, Schwartz M (2015) Immunization with a Myelin-Derived Antigen Activates the Brain's Choroid Plexus for Recruitment of Immunoregulatory Cells to the CNS and Attenuates Disease Progression in a Mouse Model of ALS. The Journal of neuroscience: the official journal of the Society for Neuroscience 35: 6381-6393
Kunis G, Baruch K, Rosenzweig N, Kertser A, Miller O, Berkutzki T, Schwartz M (2013) IFN-gamma-dependent activation of the brain's choroid plexus for CNS immune surveillance and repair. Brain: a journal of neurology 136: 3427-3440
Kotsakis A, Harasymczuk M, Schilling B, Georgoulias V. Argiris A. Whiteside T L (2012) Myeloid-derived suppressor cell measurements in fresh and cryopreserved blood samples. Journal of immunological methods 381: 14-22
Linch, S. N., McNamara, M. J., and Redmond, W. L. (2015). OX40 Agonists and Combination Immunotherapy: Putting the Pedal to the Metal. Front. Oncol. 5, 34.
Liu Y, Wang L, Predina J, Han R, Beier U H, Wang L C, Kapoor V, Bhatti T R. Akimova T. Singhal S, Brindle P K. Cole P A, Albelda S M. Hancock W W (2013) Inhibition of p300 impairs Foxp3(⁺) T regulatory cell function and promotes antitumor immunity. Nature medicine 19: 1173-1177
Marabelle A, Kohrt H, Sagiv-Barfi 1, Ajami B, Axtell R C, Zhou G, Rajapaksa R, Green M R, Torchia J, Brody J. Luong R, Rosenblum M D. Steinman L, Levitsky H I. Tse V, Levy R (2013) Depleting tumor-specific Tregs at a single site eradicates disseminated tumors. The Journal of clinical investigation 123: 2447-2463.
Marsh S E, Abud E M. Lakatos A, Karimzadeh A, Yeung S T, Davtyan H, et al. The adaptive immune system restrains Alzheimer's disease pathogenesis by modulating microglial function. Proc Natl Acad Sci USA. 2016 Mar. 1:113(9):E1316-25.
Mesquita S D, Ferreira A C, Gao F, Coppola G, Geschwind D H, Sousa J C, et al. The choroid plexus transcriptome reveals changes in type I and II interferon responses in a mouse model of Alzheimer's disease. Brain Behav Immun. 2015 October; 49:280-92.
Oakley H, Cole S L, Logan S, Maus E, Shao P, Craft J, Guillozet-Bongaarts A, Ohno M, Disterhoft J, Van Eldik L, Berry R, Vassar R (2006) Intraneuronal beta-amyloid aggregates, neurodegeneration, and neuron loss in transgenic mice with five familial Alzheimer's disease mutations: potential factors in amyloid plaque formation. The Journal of neuroscience: the of official journal of the Society for Neuroscience 26: 10129-10140
Ohaegbulam K C, Assal A, Lazar-Molnar E. Yao Y, Zang X (2015) Human cancer immunotherapy with antibodies to the PD-1 and PD-L1 pathway. Trends in molecular medicine 21: 24-33.
Puleston D J, Baixauli F. Sanin D E, Edwards-Hicks J, Villa M, Kabat A M, et al. Polyamine metabolism is a central determinant of helper T cell lineage fidelity. Cell. 2021 Jun. 25.
Qin A, Wen Z, Zhou Y, Li Y, Li Y, Luo J, Ren T, Xu L (2013) MicroRNA-126 regulates the induction and function of CD4(⁺) Foxp3(⁺) regulatory T cells through P13K/AKT pathway. Journal of cellular and molecular medicine 17: 252-264
Reagan-Show et al. (2007). Dose translation from animal to human studies revisited. The FASEB Journal 22:659-661).
Reisa A. Sperling et al. “Toward defining the preclinical stages of Alzheimer's disease: Recommendations from the National Institute on Aging—Alzheimer's Association workgroups on diagnostic guidelines for Alzheimer's disease.” Alzheimer's & Dementia: The Journal of the
Roy S, Bank S, Banerjee S, Bhuniya A, Pal S, Basu P. Biswas J, Goswami S, Chakrabortv T. Bose A, Baral R (2013) Neem leaf glycoprotein overcomes indoleamine 2,3 dioxygenase mediated tolerance in dendritic cells by attenuating hyperactive regulatory T cells in cervical cancer stage 111B patients. Human immunology 74: 1015-1023
Schwartz M, Baruch K (2014a) Breaking peripheral immune tolerance to CNS antigens in neurodegenerative diseases: boosting autoimmunity to fight-off chronic neuroinflammation. Journal of autoimmunity 54: 8-14
Schwartz M, Baruch K (2014b) The resolution of neuroinflammation in neurodegeneration: leukocyte recruitment via the choroid plexus. The EMBO journal 33: 7-22.
Smith P M, Howitt M R, Panikov N, Michaud M, Gallini C A, Bohlooly Y M, Glickman J N. Garrett W S (2013) The microbial metabolites, short-chain fatty acids, regulate colonic Treg cell homeostasis. Science 341: 569-573.
Tarragó M G, Chini C C S, Kanamori K S, Warner G M, Cande A, de Oliveira G C, et al. A Potent and Specific CD38 Inhibitor Ameliorates Age-Related Metabolic Dysfunction by Reversing Tissue NAD+ Decline. Cell Metab. 2018 May 1, 27(5):1081-1095.e10.
Ward F J. Dahal L N, Wijesekera S K, Abdul-Jawad S K, Kaewarpai T, Xu H, Vickers M A, Barker R N (2013) The soluble isoform of CTLA-4 as a regulator of T-cell responses. European journal of immunology 43: 1274-1285
Weber J S, Kudchadkar R R. Yu B, Gallenstein D, Horak C E, Inzunza H D, Zhao X, Martinez A J, Wang W, Gibney G, Kroeger J, Eysmans C, Samaik A A, Chen Y A (2013) Safety, efficacy, and biomarkers of nivolumab with vaccine in ipilimumab-refractory or -naive melanoma. Journal of clinical oncology: official journal of the American Society of Clinical Oncology 31: 4311-4318
Zhao L, Sun L. Wang H, Ma H, Liu G, Zhao Y (2007) Changes of CD4⁺CD25⁺Foxp3⁺ regulatory T cells in aged Balb/c mice. Journal of leukocyte biology 81: 1386-1394
Zheng H, Fridkin M, Youdim M (2015) New approaches to treating Alzheimer's disease. Perspectives in medicinal chemistry 7: 1-8

1. A method for early detection or diagnosis of a neurodegenerative disease, disorder, or condition in a subject at risk of developing or suspected of having the neurodegenerative disease, disorder, or condition, the method comprising measuring in a blood sample obtained from the subject or a fraction thereof the levels of at least one biomarker selected from the group consisting of CD38⁺ peripheral blood mononuclear cells (PBMCs), trigonelline, GLUT1 expression in CD4⁺ T cells, Tₕ2, Tₕ2/Tₕ1 ratio, naïve T cells, adenosine, allose, and HLA-DR T cells,
wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that the subject is likely developing, or affected by, said neurodegenerative disease, disorder, or condition.

2. The method of claim 1, wherein the at least one biomarker comprises CD38⁺ PBMCs.

3. The method of claim 2, wherein the increased level of CD38⁺ PBMCs compared to the respective reference for CD38⁺ PBMCs level is increased by at least about 10%, 15%, 20%, 25%, 30%, 40%, 50%, 60%, 70%, 80%, 90%, or 100%.

4. The method of claim 2, wherein the CD38⁺ PBMCs are CD38⁺ CD4⁺ or CD38⁺ CD8⁺ T cells, and their level is calculated by measuring the levels of (1) CD38⁺ CD4⁺ cells and (2) total CD4⁺ cells, and calculating the proportion of (1) out of (2), or measuring the levels of (1) CD38⁺ CD8⁺ cells and (2) total CD8⁺ cells, and calculating the proportion of (1) out of (2), respectively.

5. The method of claim 4, wherein the at least one biomarker comprises CD38⁺ CD4⁺ T cells and CD38⁺ CD8⁺ T cells.

6. The method of claim 4, wherein the respective reference for CD38⁺ CD4⁺ T cells out of total CD4⁺ T cells is between about 35% and about 40%, and the respective reference for CD38⁺ CD8⁺ T cells out of total CD8⁺ T cells is between about 15% and about 25%.

7. The method of claim 1, wherein the at least one biomarker comprises Tₕ2 cells and/or Tₕ2/Tₕ1 ratio.

8. The method of claim 7, wherein the level of the Tₕ2 cells is measured by measuring the level of CXCR5⁻CCR6⁻CXCR3⁻CD⁴⁺ cells, and/or the Tₕ2/Tₕ1 ratio is calculated by measuring the level of (1) CXCR5⁻CCR6⁻CXCR3⁻CD4⁺cells (Tₕ2), and (2) CXCR5⁻CCR6⁻CXCR3⁺CD4⁺ cells (Tₕ1), and calculating the proportion of (1) out of (2).

9. The method of claim 7, wherein the increased Tₕ2/Tₕ1 ratio is increased by at least about 30%, 35%, 40%, 45%, 50%, 60%, or 70% compared to the respective reference for Tₕ2/Tₕ1 ratio.

10. The method of claim 7, wherein the respective reference for Tₕ2/Tₕ1 ratio is at least about 10, 11, 12, 13, or 14.

11. The method of claim 1, wherein the at least one biomarker comprises naïve CD4⁺ T cells level.

12. The method of claim 11, wherein the naïve CD4⁺ T cells level is calculated by measuring the level of (1) CCR7⁺CD45RO⁻CD45RA⁺ (naïve) CD4⁺ T cells, and (2) total CD4⁺ T cells, and calculating the proportion of (1) out of (2).

13. The method of claim 11, wherein the increased level of naïve CD4⁺ T cells compared to the respective reference is increased by at least about 30%, 35%, 40%, 45, or 50%.

14. The method of claim 1, wherein the at least one biomarker comprises GLUT1 expression in CD4⁺ T cells.

15-17. (canceled)

18. The method of claim 14, wherein the at least one biomarker comprises CD38+ PBMCs and GLUT1 expression in CD4⁺ T cells.

19. The method of claim 7, wherein the at least one biomarker comprises CD38⁺ PBMCs and Tₕ2/Tₕ1 ratio.

20. (canceled)

21. The method of claim 18, wherein increased levels of CD38+ PBMCs compared to a respective reference and decreased levels of GLUT1 expression in CD4⁺ T cells as compared to a respective reference indicate that the subject is likely developing, or affected by, said neurodegenerative disease, disorder, or condition.

22. (canceled)

23. The method of claim 1, wherein said neurodegenerative disease, disorder, or condition is selected from the group consisting of Alzheimer's disease (AD), amyotrophic lateral sclerosis (ALS), Parkinson's disease, Huntington's disease, primary progressive multiple sclerosis; secondary progressive multiple sclerosis, attention deficit disorder (ADD), corticobasal degeneration, Creutzfeldt-Jakob disease (CJD), Rett syndrome, a retinal degeneration disorder selected from the group consisting of age-related macular degeneration and retinitis pigmentosa; anterior ischemic optic neuropathy; glaucoma; uveitis; depression; trauma-associated stress or post-traumatic stress disorder, frontotemporal dementia (FTD), Lewy body dementias, mild cognitive impairments, posterior cortical atrophy, primary progressive aphasia and progressive supranuclear palsy.

24-26. (canceled)

27. A method of assessing efficacy of a treatment by an immune checkpoint modulator in a subject diagnosed with a neurodegenerative disease, disorder, or condition, said method comprising:
obtaining a first blood sample from the subject;
administering the immune checkpoint modulator to the subject;
obtaining a second blood sample from the subject;
measuring in the first blood sample or a fraction thereof and in the second blood sample or fraction thereof the levels of at least one biomarker selected from the group consisting of CD38⁺ peripheral blood mononuclear cells (PBMCs), trigonelline, GLUT1 expression in CD4⁺T cells, Tₕ2, Tₕ2/Tₕ1 ratio, naïve T cells, adenosine, allose, and HLA-DR T cells, and
comparing the level of the at least one biomarker in the first blood sample with the level of the biomarker in the second blood sample;
wherein a decreased level of at least one of CD38⁺ PBMCs, Tₕ2, Tₕ2/Tₕ1 ratio, naïve T cells, and adenosine, and/or an increased level of at least one of trigonelline, GLUT1 expression in CD4⁺ T cells, allose, and HLA-DR T cells in the second blood sample compared to the first blood sample indicates that the treatment by an immune checkpoint modulator is likely effective.

28. A method for treating or preventing a neurodegenerative disease, disorder, or condition in a subject, said method comprising measuring in a blood sample or fraction thereof the levels of at least one biomarker selected from the group consisting of CD38⁺ peripheral blood mononuclear cells (PBMCs), trigonelline, GLUT1 expression in CD4⁺T cells, Tₕ2, Tₕ2/Tₕ1 ratio, naïve T cells, adenosine, allose, and HLA-DR T cells,
wherein an increased level of at least one of CD38⁺ PBMCs, Tₕ2, Tₕ2/Tₕ1 ratio, naïve T cells, and adenosine, as compared to a respective reference, and/or a decreased level of at least one of trigonelline, GLUT1 expression in CD4⁺ T cells, allose, and HLA-DR T cells as compared to a respective reference, indicates that the subjects is likely developing, or is affected by, the neurodegenerative disease, disorder, or condition, and
the method further comprising administering an immune checkpoint modulator to the subject which is likely developing, or is affected by, the neurodegenerative disease, disorder, or condition, thereby treating the subject.

29. (canceled)
